(19) United States
(12) Patent Application Publication
(10) Pub. No.: US 2009/0012541 A1
(43) Pub. Date: Jan. 8, 2009

(54) EXPANDABLE FASTENER SYSTEM WITH FLOWER PETAL-SHAPED RETENTION ELEMENTS

(75) Inventors: Terry Dahl, Santa Barbara, CA (US); Gregg Sutton, Plymouth, MN (US); Mitchell Dann, Wilson, WY (US); Greg Fluet, Jackson, WY (US); James Wright, Carpinteria, CA (US)

Correspondence Address: KNOBBE MARTENS OLSON & BEAR LLP, 2040 MAIN STREET, FOURTEENTH FLOOR, IRVINE, CA, 92614, US

(73) Assignee: VALENTX, INC., Carpinteria, CA (US)

(21) Appl. No.: 12/137,473

(22) Filed: Jun. 11, 2008

Related U.S. Application Data
(60) Provisional application No. 60/943,304, filed on Jun. 11, 2007.
(60) Provisional application No. 61/033,385, filed on Mar. 3, 2008.
(60) Provisional application No. 61/042,190, filed on Apr. 3, 2008.

(51) Int. Cl. A61B 17/08; A61M 29/00; A61B 17/04

(52) U.S. Cl. 606/151; 606/192; 606/228; 606/232

(57) ABSTRACT

Disclosed herein are various devices and methods that can be utilized independently or in conjunction with each other for endoscopic delivery of a wide ranges of medical devices, such as, for example, an endoscopic gastrointestinal bypass sleeve with an attachment cuff. Three primary components of the system include a space-creating device; an expandable fastener system with flower petal-shaped retention elements; and an endoscopic curved needle driver system.

### PRIORITY CLAIM

The present application claims priority under 35 U.S.C. § 119(e) to U.S. Provisional Application Nos. 60/943,304 entitled “ENDOSCOPIC CURVED NEEDLE DRIVER” and filed Jun. 11, 2007; 61/033,385 entitled “EXPANDABLE FASTENER SYSTEM WITH FLOWER PETAL-SHAPED RETENTION ELEMENTS” and filed Mar. 3, 2008; and 61/042,190 entitled “DEVICES AND METHODS FOR CREATION OF A WORKING SPACE IN A BODY LUMEN”, filed Apr. 3, 2008. All three of the aforementioned priority applications are hereby incorporated by reference in their entirety.

### SUMMARY OF THE INVENTION

Disclosed herein is an expandable fastener for securing a device transmurally to a surface of a tissue wall, according to some embodiments of the invention. The fastener can include a first retention element comprising a plurality of petals extending from a central hub. The plurality of petals has a total surface area. The first retention element can be movable from a compressed configuration for delivery to the surface of the tissue wall and an expanded configuration for engaging tissue. The first retention element defines an effective footprint of the first retention element. The effective footprint is defined by the smallest diameter circle circumscribing the plurality of petals while the first retention element is in its expanded configuration. In some embodiments, the total surface area of the plurality of petals is no more than about 80%, 70%, or 60% of the area of the effective footprint of the first retention element. In some embodiments, the expandable fastener further includes a tension element having an elongate body, a proximal end, and a distal end. The tension element can be operably attached to the central hub. In some embodiments, the smallest diameter circle circumscribing the plurality of petals has a diameter of between about 0.10 inches and 0.50 inches. In some embodiments, the first retention element comprises between 2 and 10 petals. In some embodiments, the plurality of petals can be formed from one or more wires, the one or more wires having a diameter of between about 0.001 inch and 0.050 inches. The plurality of petals can include a tissue-ingrowth material. The tension element can have a length that is at least about 100% of the thickness of the tissue wall. In some embodiments, the fastener can include a second retention element operably connected to the proximal end of the tension element. The tension element can be a suture, a T-tag, or a button in some embodiments. The petals of a retention element can be configured to be independently movable with respect to one another.

Also disclosed herein in some embodiments is a method of attaching a device transmurally through a tissue wall of a body lumen having a serosal surface and a mucosal surface. The method can include the steps of positioning an endoscope within a body lumen, the endoscope comprising a working channel housing a needle driver therein; the needle driver comprising a working channel with a needle with a proximal zone and a distal zone housed therein, the distal zone of the needle driver having a first straightened configuration while within the working channel of the needle driver and a second curved unstressed configuration, the needle comprising a lumen housing a fastening system comprising a first retention element, a second retention element, and a tension element operably connected to the first retention element and the second retention element; actuating the needle driver such that at least a portion of the distal zone of the needle is outside of the working channel of the needle driver and assumes its second curved unstressed configuration; advancing the needle through the luminal wall such that an end of the distal zone of the needle is positioned on the serosal side of the wall; releasing the first retention element on the serosal side of the tissue wall; withdrawing the distal end of the needle driver such that it is positioned on the mucosal side of the tissue wall; and releasing the second retention element on the mucosal side of the tissue wall to secure the device to the tissue wall. The method can also include the step of dilating the body lumen to create an endoscopic working space. Dilating the body lumen is accomplished using an expandable stent, such as by expanding a proximal diameter of the expandable stent to greater than a distal diameter of the expandable stent. The first retention element can include a plurality of petals operably connected to a central hub, and include between 4 and 10 petals. In some embodiments, the second retention element could include a T-tag or a button. The device to be attached could be an attachment cuff, which in turn could be operably attached to a gastrointestinal bypass sleeve. The body lumen could be, in some embodiments, the esophagus or the stomach. The tissue wall could be the wall of the gastroesophageal junction.

Also disclosed herein according to some embodiments is a needle driver for delivering a tissue fastener through a tissue side wall, comprising: an elongate body having a lumen therethrough and a proximal handle portion; a needle configured to reside within the lumen of the needle driver, the needle having a proximal zone and a distal zone, the distal zone of the needle having a first straightened configuration while within a working channel of the needle driver and a second unstressed curved configuration, the needle having a lumen therethrough; a sheath configured to house the needle; a stylet configured to house a tissue fastener; and a first actuator for moving the needle axially relative to the sheath; and a second actuator for moving the stylet axially relative to the needle. The length of the distal zone of the needle is between about 1-2 inches in some embodiments. The distal zone could have an arc angle in its second unstressed curved configuration of between about 40 degrees and 70 degrees in some embodiments.

Also disclosed herein according to some embodiments is an endoscopic delivery kit, comprising: a needle driver comprising a needle having a proximal zone and a distal zone, the distal zone of the needle having a first straightened configuration while within a working channel of the needle driver and a second curved unstressed configuration, the needle comprising a lumen; and a fastening system housed within the lumen of the needle, the fastening system comprising a first retention element, a second retention element, and a tension element operably connected to the first retention element and the second retention element. The endoscopic delivery kit could also include a space-creating stent comprising a plurality of interconnected struts joined together such that an inner lumen is formed therethrough, the struts having a substantially straight distal portion and a curved proximal portion; wherein at least one of the struts comprise an eyelet on its proximal portion, the eyelet configured to house a control element therethrough configured to actuate a proximal diameter of the stent from a first larger diameter to a second smaller diameter.

Also disclosed herein is a space-creating stent for creating a working space in a body lumen, comprising a plurality of interconnected struts joined together such that an inner lumen is formed therethrough, the struts having a substantially straight distal portion and a curved proximal portion; wherein at least one of the struts comprise an eyelet on its proximal portion, the eyelet configured to house a control element therethrough configured to change a proximal diameter of the stent from a first larger diameter to a second smaller diameter. The stent could be formed from a wire in some embodiments. In some embodiments, each of the proximal portions of the struts comprise an eyelet. In some embodiments, at least one of the struts comprise an eyelet on its distal portion. In some embodiments, each of the distal portions of the struts comprise an eyelet. The stent could further include a plurality of barbs on an outer surface of the stent.

In some embodiments, also disclosed is a system for creating a working space in a body lumen, comprising: a stent comprising a plurality of interconnected struts joined together such that an inner lumen is formed therethrough, the struts having a substantially straight distal portion and a curved proximal portion; wherein at least two of the struts comprise an eyelet on its proximal portion, the eyelet configured to house a control element therethrough configured to actuate a proximal diameter of the stent from a first larger diameter to a second smaller diameter; and a control catheter operably attached to and configured to actuate the control element.

### BRIEF DESCRIPTION OF THE DRAWINGS

FIGS. 1A-1D illustrate various components of a delivery system for attaching a gastrointestinal bypass sleeve with an attachment cuff, according to one embodiment of the invention.

FIG. 2 illustrates a wire that can be used to form a space-creating stent within a body lumen, according to one embodiment of the invention.

FIG. 3A illustrates a top view of a space-creating stent with its proximal end in an expanded configuration, according to one embodiment of the invention.

FIG. 3B illustrates a side view of the space-creating stent of FIG. 3A.

FIG. 3C illustrates a space-creating stent with its proximal end in a collapsed configuration, according to one embodiment of the invention.

FIG. 3D illustrates a side view of the space-creating stent of FIG. 3C.

FIG. 3E illustrates a perspective view of a space-creating stent, according to one embodiment of the invention.

FIG. 4 illustrates a control catheter for actuating a control element configured to adjust the diameter of a space-creating device, according to one embodiment of the invention.

FIGS. 5-9 are various cross-sectional views of the control catheter of FIG. 4.

FIGS. 10A-10C illustrate releasable connectivity of an introducer plug with an overtube introducer tip and a control catheter, according to one embodiment of the invention.

FIG. 11 illustrates one embodiment of a control catheter with a plurality of suture loops.

FIGS. 12A-B illustrate end views of an embodiment of a first retention element with a plurality of retention surfaces.

FIG. 13 is a transverse sectional view through line A-A of the retention element of FIG. 12A.

FIG. 14 is a close-up view of circled area B of FIG. 13, illustrating tension element with surfaces to secure the tension element with respect to the hub.

FIG. 15 is a perspective view of a retention element similar to retention element illustrated in FIG. 12A, illustrating a plurality of petals operably connected to distal hub, which in turn is operably connected to tension element, according to one embodiment of the invention.

FIG. 16A is a side view and FIG. 16B is an angled perspective view of a fastener system, according to one embodiment of the invention.

FIG. 16C is an end view of a retention element, according to one embodiment of the invention.

FIG. 17A is a perspective view of one embodiment of a retention element with two petals operably connected to a central hub.

FIG. 17B is a retention element similar to the retention element of FIG. 17A, with a lower profile hub that may be axially in-line or substantially axially in-line with a plane of the long axis of the petals.

FIG. 17C is a top view of the retention element of FIG. 17B.

FIG. 17D is a perspective view of an embodiment of a retention element that includes three petals, with a lower profile hub as previously noted.

FIG. 17E is a top view of the retention element of FIG. 17D.

FIG. 18 illustrates an embodiment of a fastener system, housed within a delivery cannula.

FIG. 19 illustrates another embodiment of a fastener system including a proximal retention element that can be located outside of the delivery cannula during delivery.

FIG. 20 illustrates a hollow curved needle partially enveloped by a sheath for endoscopically delivering a fastening element, according to one embodiment of the invention.

FIG. 21 is a perspective view that illustrates a deployment system for a curved needle driver, according to one embodiment of the invention.

FIG. 22 is a cut-away view of the system shown in FIG. 21.

FIGS. 23-30 schematically illustrate steps of a method for attaching a attachment cuff with gastrointestinal bypass sleeve using a space-creating stent, expandable fastener, and curved needle driver, according to one embodiment of the invention.

FIGS. 31-33 schematically illustrate another method of creating a working space within a gastrointestinal lumen, according to one embodiment of the invention.

### DETAILED DESCRIPTION OF PREFERRED EMBODIMENTS

Disclosed herein are various devices and methods that can be utilized during endoscopic delivery of a wide ranges of medical devices, such as, for example, an endoscopic gastrointestinal bypass sleeve with an attachment cuff Three primary components of the system will be described herein: (1) a space-creating device; (2) an expandable fastener system with flower petal-shaped retention elements; and (3) an endoscopic curved needle driver.

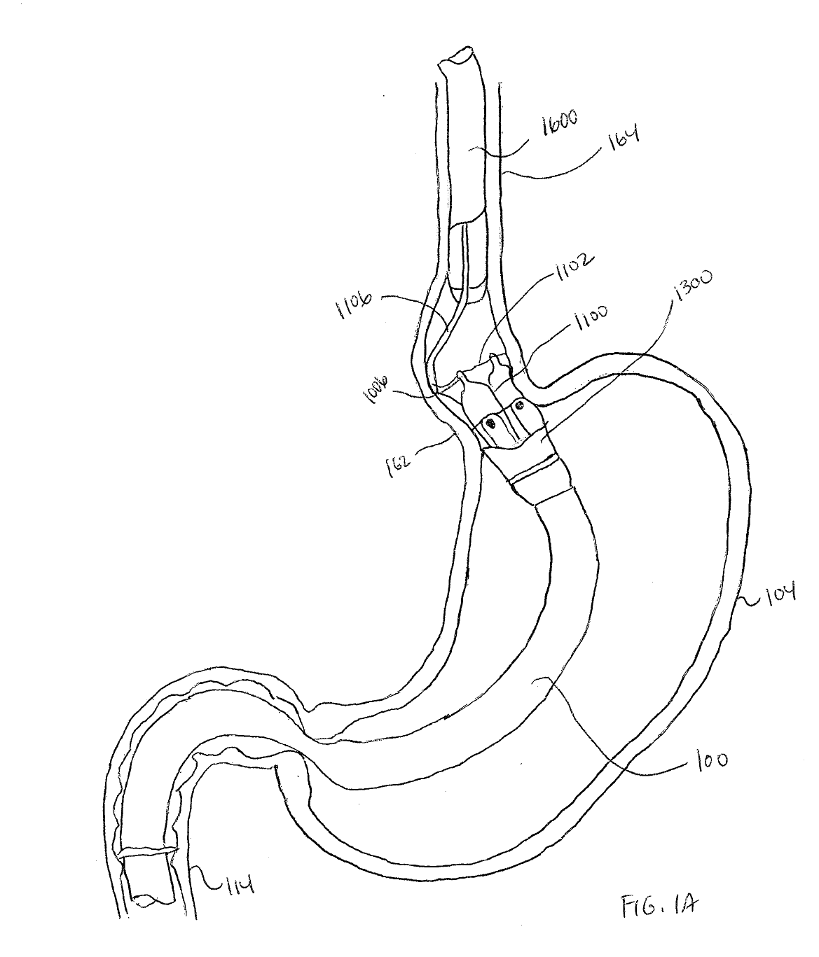

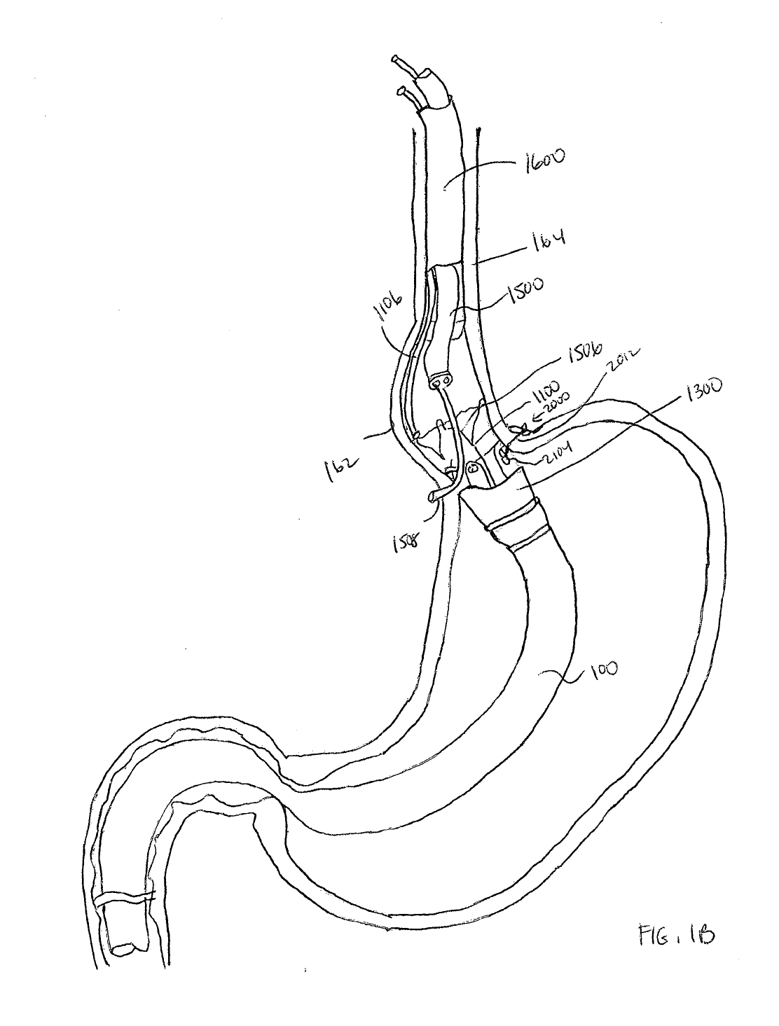

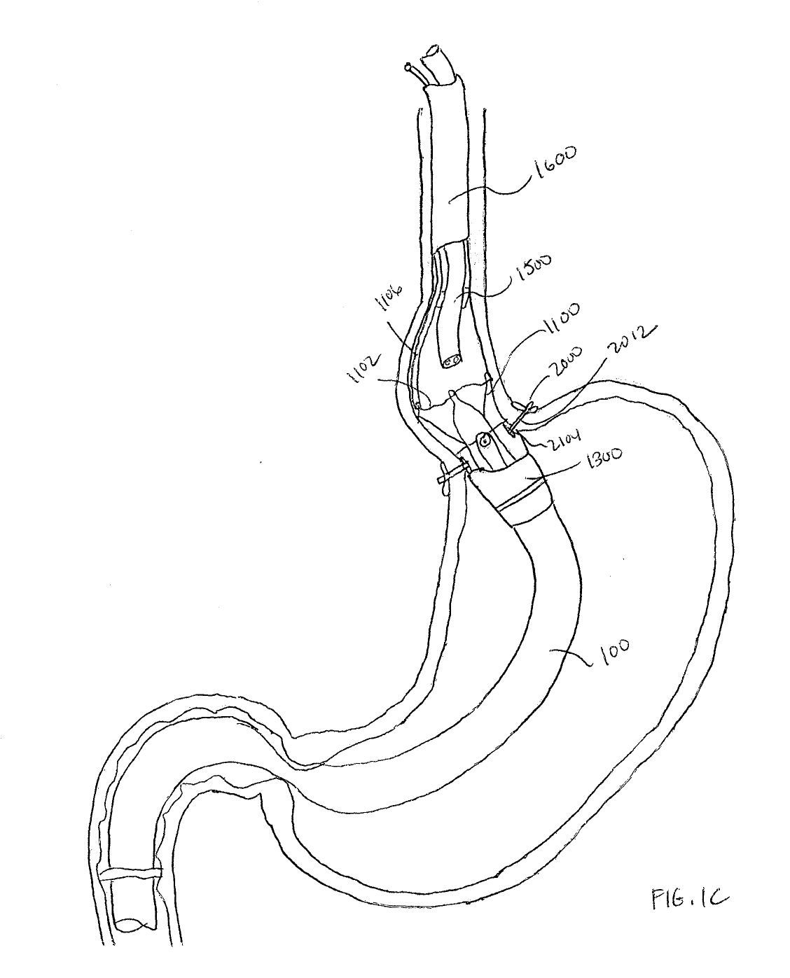

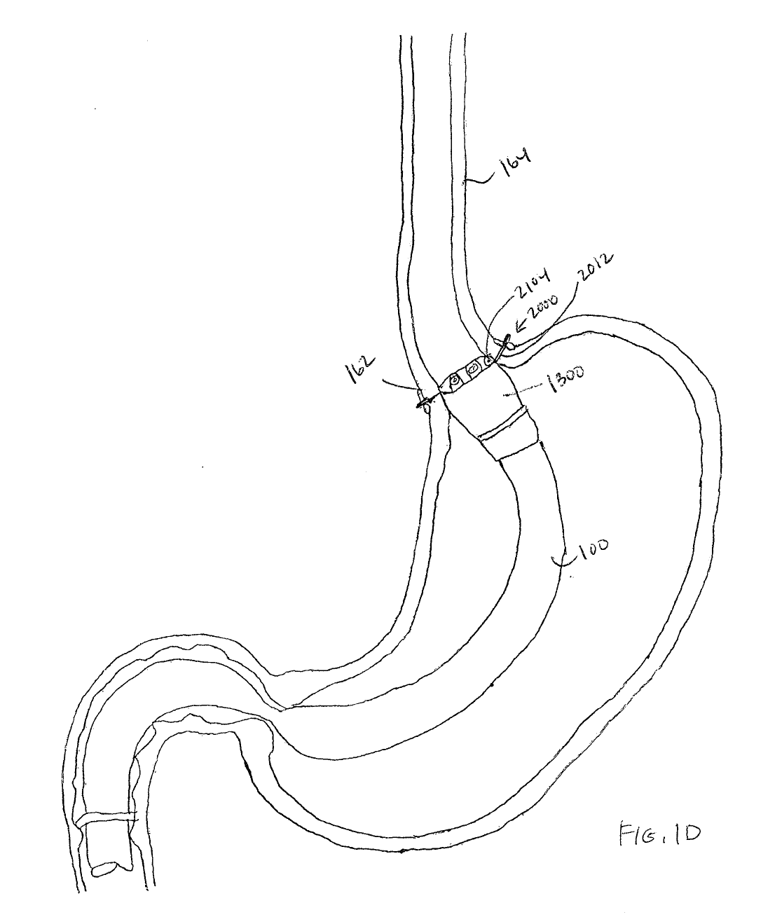

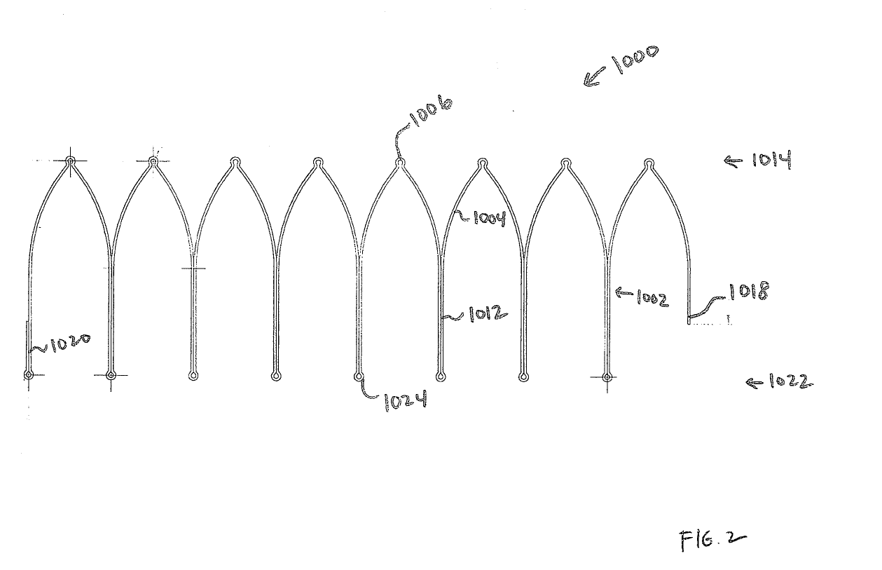

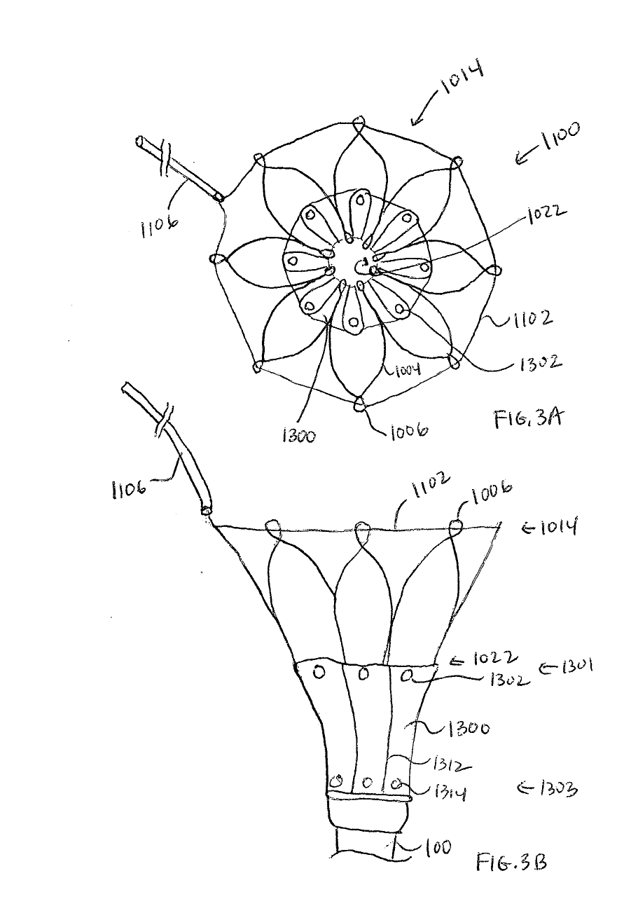

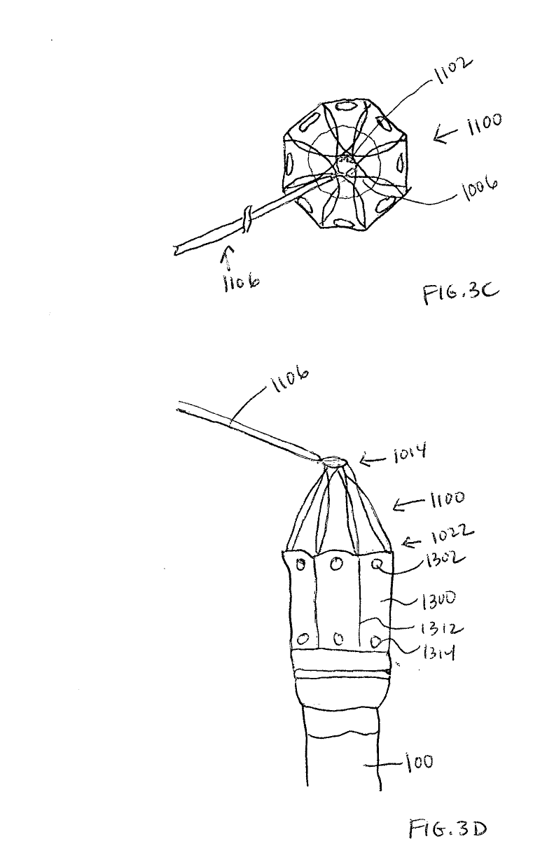

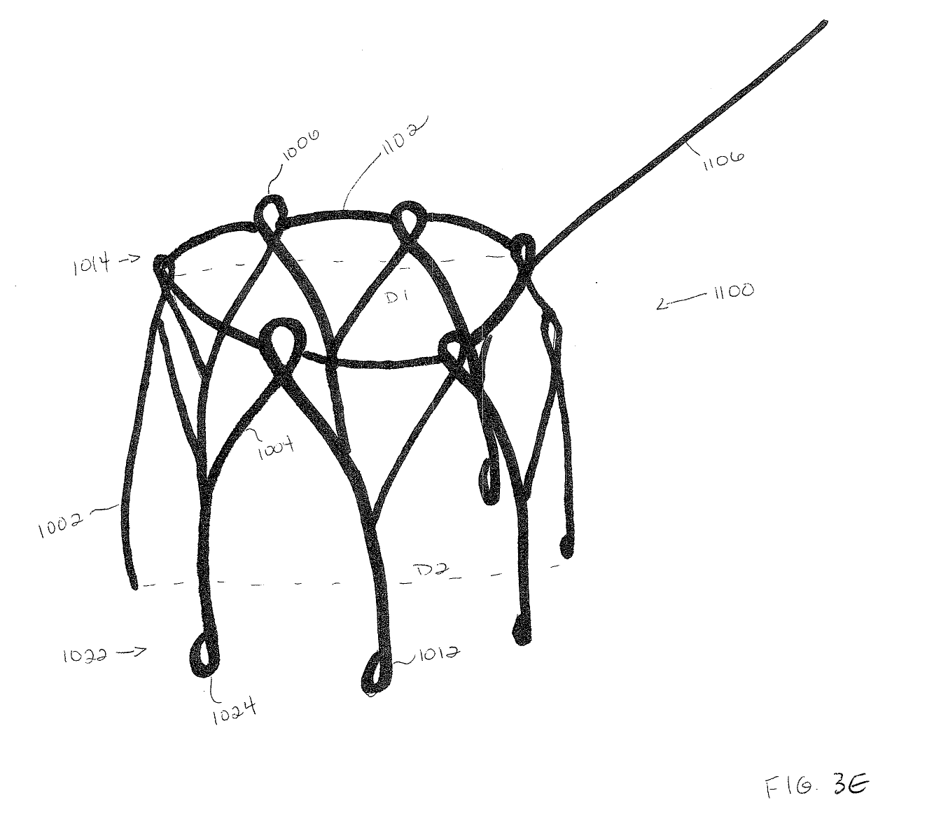

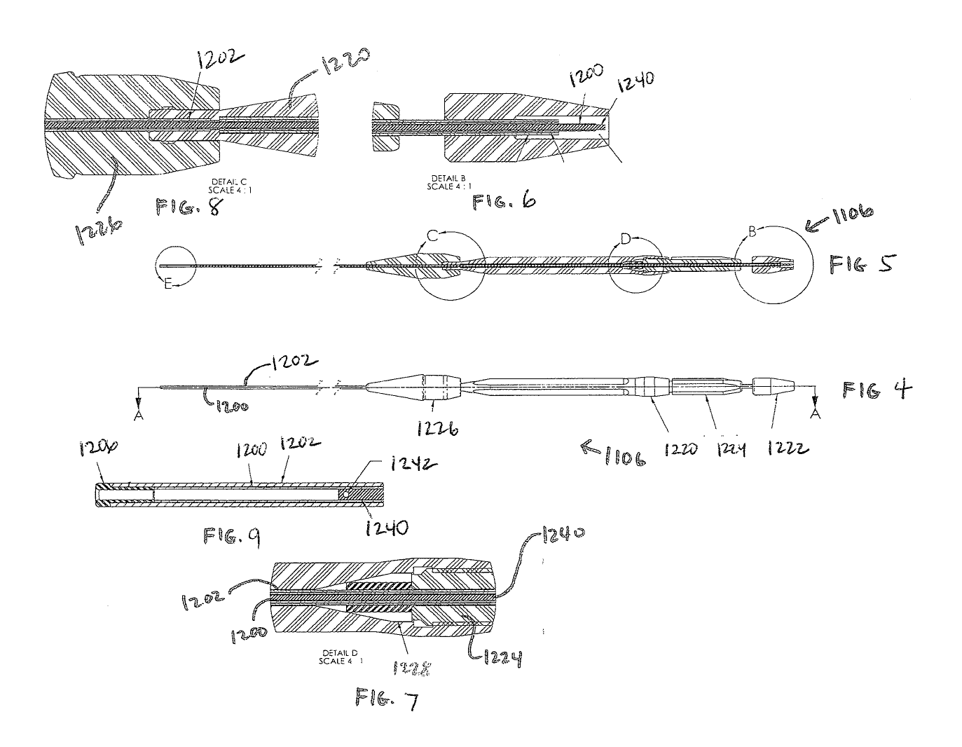

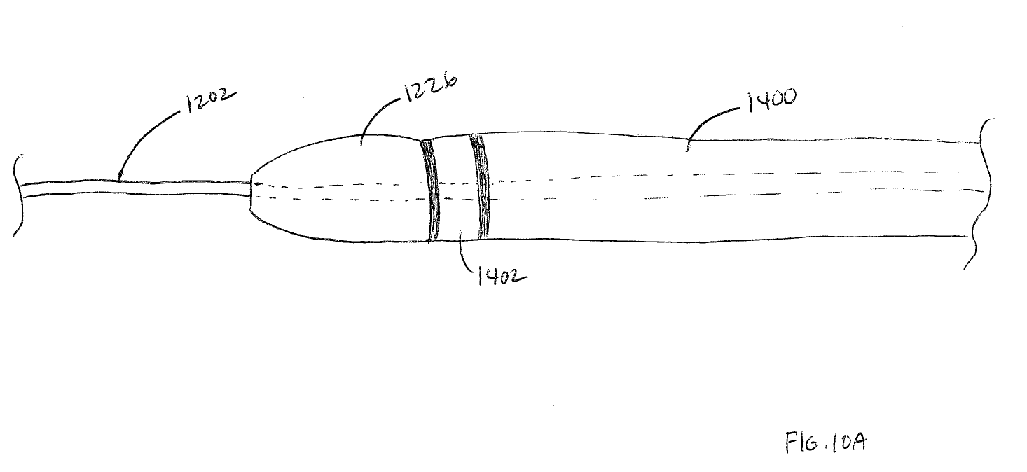

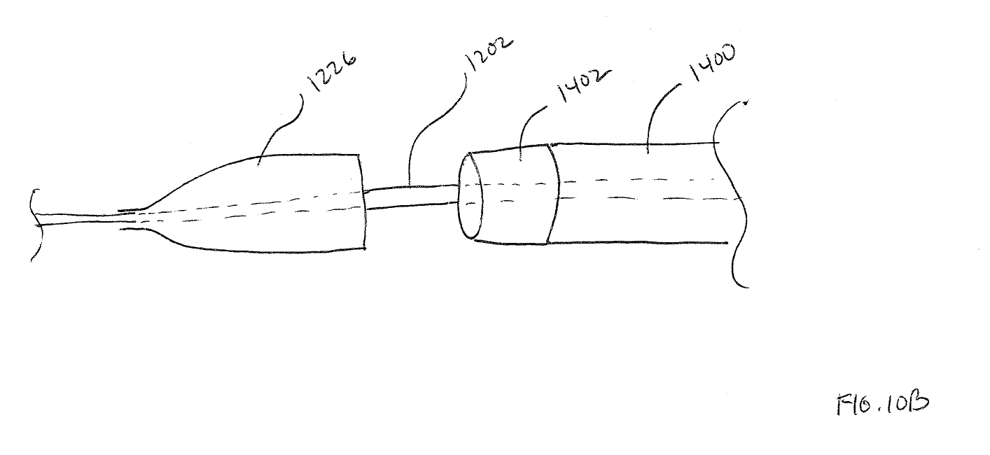

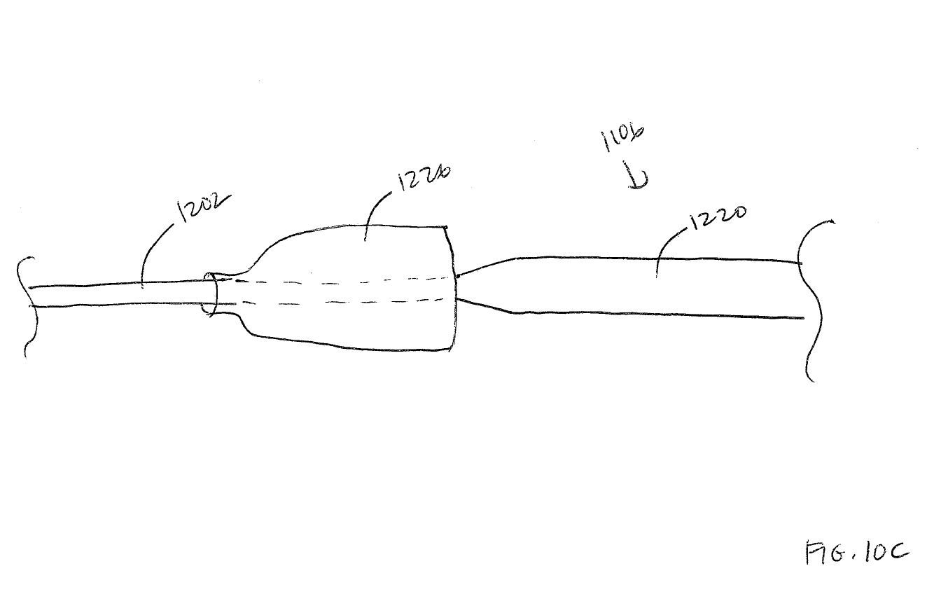

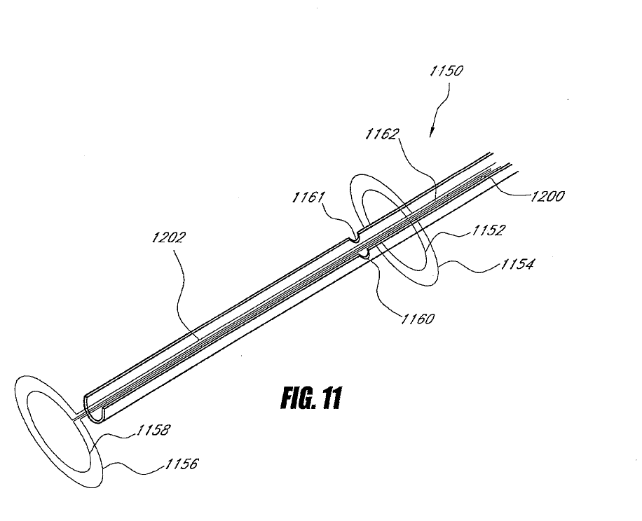

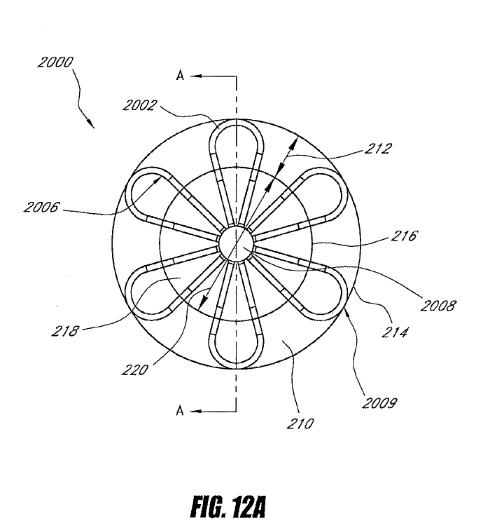

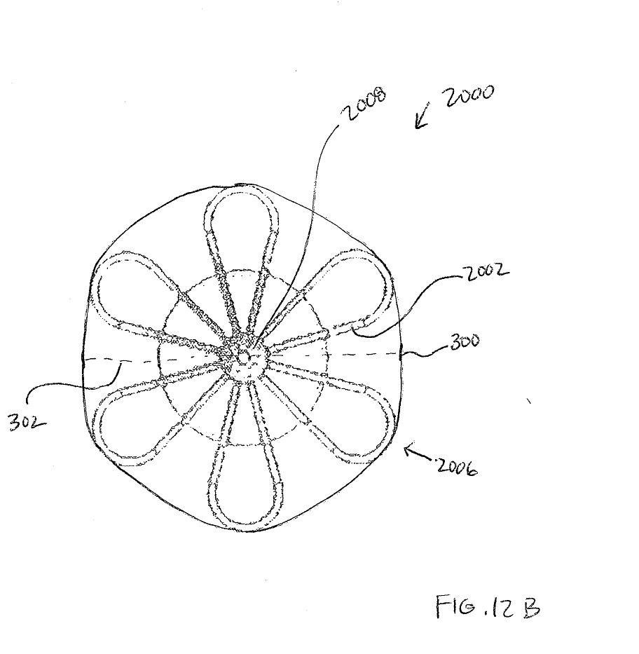

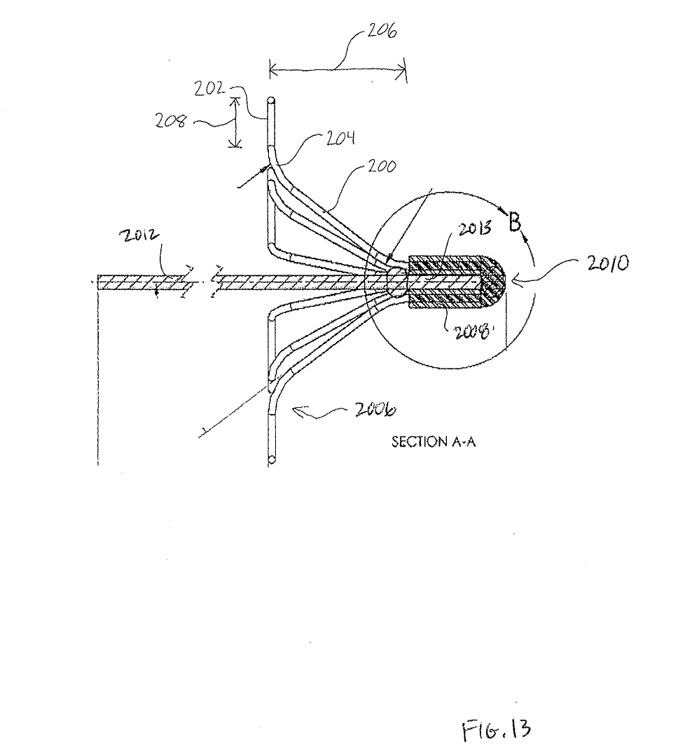

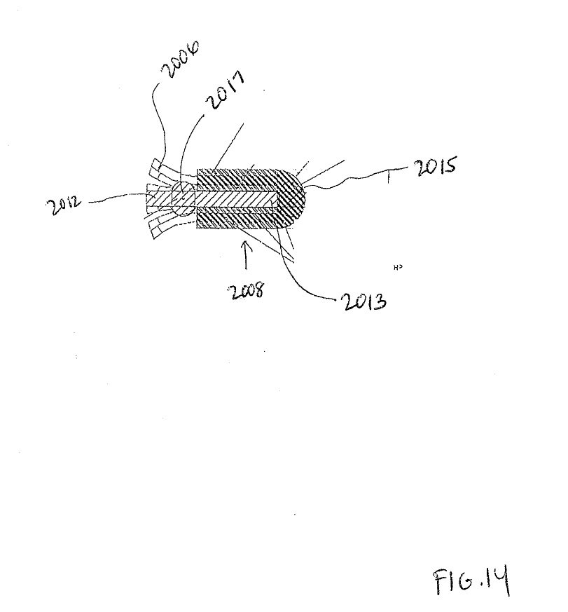

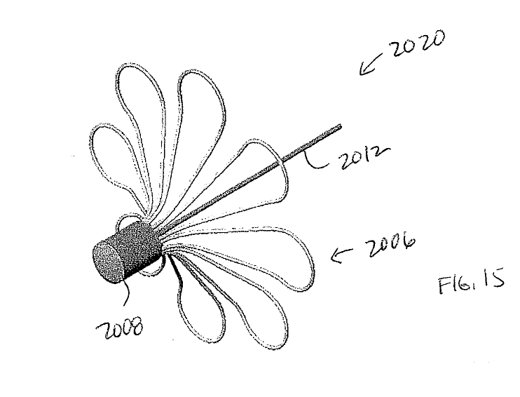

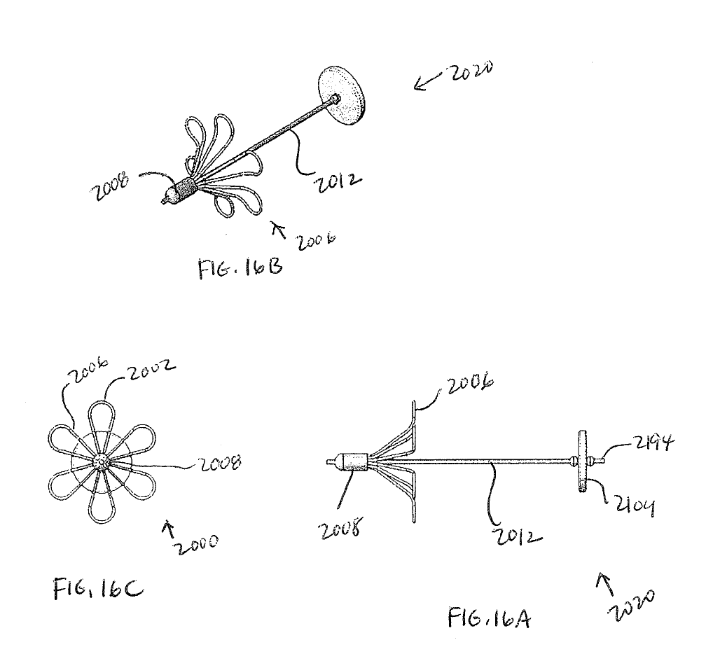

A brief overview of the three primary components of the system in the context of attaching an attachment cuff with a gastrointestinal bypass sleeve through the wall of the gastroesophageal junction follows. Various other details of the three primary components as well as a variety of other uses for the components either in concert or separately are described later in the application. As illustrated in FIG. 1A, the space creating device can include a stent 1100. The stent 1100 can be used to create and stably maintain an endoscopic working space within a body lumen, such as in the esophagus 164 or at the gastroesophageal junction 162, for example, to manipulate tissue. The stent 1100 can include eyelets 1006 on its proximal ends and/or distal ends (not shown) to receive a control element 1002, such as a suture, therethrough. The control element 1102 can be actuated by a control catheter 1106 to selectively collapse or expand the proximal and/or distal ends of the stent and thus adjust the dimensions of the working space according to the desired clinical result. After deployment and expansion of the stent 1100 in the desired anatomical location, an expandable fastener system (not shown in FIG. 1A) can be loaded into a delivery cannula, which can be a needle driver comprising a needle 1506 with a curved distal tip portion 1508 as shown in FIG. 1B. The expandable fastener system can be used to secure the bypass sleeve 100 transmurally through a luminal wall to a serosal surface, such as at the gastroesophageal junction 162. The expandable fastener system can include a distal retention element 2000 having a plurality of petals, and is configured to allow the retention element to provide a relatively large effective footprint for retaining the sleeve while maintaining a relatively small actual tissue-device contact area, as will be described in detail below. The distal retention 2000 element is connected to a tension element 2012 and a proximal retention element 2104 for connection to the attachment cuff 1300. The needle 1506 can include a pre-set curved distal section 1508 that is kept in a relatively straightened configuration by the walls defining a working channel of the needle driver. Upon being actuated distally, the distal zone 1508 needle takes its unstressed curved shape and can cannulate the wall of the GI tract through the serosa as shown in FIG. 1B. The distal retention element 2000 can then be ejected from the needle 1106 on the serosal side of the tissue wall, which is then withdrawn proximally and the proximal retention element 2104 ejected within the interior lumen of the attachment cuff 1300, tensioning the tension element 2012 and securing the cuff 1300 together with sleeve 100, as shown in FIG. 1C. The needle 1506 is retracted into the needle driver and the endoscope 1500 can then be removed, followed by contraction and removal of the space-creating device 1100, leaving the fastening system including proximal retention element 2104, tension element 2012, and distal retention element 2000 securing the attachment cuff 1300 and bypass sleeve 100 at the gastroesophageal junction 162 as shown in FIG. 1D.

Various features of, for example, gastrointestinal bypass sleeves, attachment cuffs, and/or toposcopic delivery methods that can be used or adapted for use with systems and methods disclosed herein can be found, for example, at U.S. patent application Ser. No. 10/698,148, filed Oct. 31, 2003, published May 13, 2004 as U.S. Patent Pub. No. 2004-0092892 A1 and entitled “APPARATUS AND METHODS FOR TREATMENT OF MORBID OBESITY” (and may be referred to herein as the “Kagan '148 application or Kagan '892 publication”); U.S. patent application Ser. No. 11/025,364, filed Dec. 29, 2004, published Aug. 11, 2005 as U.S. Patent Pub. No. 2005-0177181 A1 and entitled “DEVICES AND METHODS FOR TREATING MORBID OBESITY” (and may be referred to herein as the “Kagan '181 publication”); U.S. patent application Ser. No. 11/124,634, filed May 5, 2005, published Jan. 26, 2006 as U.S. Patent Pub. No. 2006-0020247 A1 and entitled “DEVICES AND METHODS FOR ATTACHMENT OF AN ENDOLUMENAL GASTROINTESTINAL IMPLANT” (and may be referred to herein as the “Kagan '247 publication”); U.S. patent application Ser. No. 11/400,724, filed Apr. 7, 2006, published Jan. 11, 2007 as U.S. Patent Pub. No. 2007-0010794 A1 and entitled “DEVICES AND METHODS FOR ENDOLUMENAL GASTROINTESTINAL BYPASS” (and may be referred to herein as the “Dann '794 publication”); and U.S. patent application Ser. No. 11/548,605, filed Oct. 11, 2006, published Aug. 23, 2007 as U.S. Pub. No. 2007-0198074 A1 and entitled “DEVICES AND METHODS FOR ENDOLUMENAL GASTROINTESTINAL BYPASS” (and may be referred to herein as the “Dann '605 application” or “Dann '074 publication”); and U.S. Provisional Application No. 60/943,014 filed Jun. 8, 2007 and entitled “GASTROINTESTINAL BYPASS SLEEVE AS AN ADJUNCT TO BARIATRIC SURGERY” are hereby incorporated by reference in their entireties herein, as well as any additional applications, patents, or publications noted in the specification below.

### Space-Creating Device

Various procedures are conducted in the GI tract for both diagnostic and therapeutic reasons. Most of these procedures are done under direct visualization using an endoscope, enteroscope, colonoscope or other such device.

The stomach and other lumens in the GI tract have highly mobile walls and tend to be easily displaced when acted on by a force. They are also highly muscular and expand and contract in various cycles. At any time the lumen can be open or closed, but is most often in more of a collapsed state. Pressure though the endoscope or an insufflation port is also used to create more space to view the lumen.

Pressure works well for visualization but there are conditions when its utility is limited. In addition, if there is pressure in the area around the lumen, for example if there is insufflation for a laparoscopic procedure, the ability to use pressure through the endoscope is compromised. Space-creating devices as described herein can make a stable working space so a specific location to transect the wall of the stomach can be identified and accurately targeted. The space creator advantageously eliminates the need for air or CO2 insufflation to create and maintain a working space. This is potentially a simpler and more consistent method for space creation, as there is no need to prevent leakage of the insufflating gas. The dimensions of the space can remain relatively constant, without having to rely on a regulated pressure system to maintain the space.

Endoscopes have a limited amount of steerability and it is challenging to access the walls of the lumen with standard endoscopic working channels that are aligned along the main axis of the endoscope because these are by nature positioned more coaxially with the main axis of the lumen. There are endoscopes with side-firing working channels and these are often used for procedures such as ERCP. However, these still do not address the issue of holding the treatment site fixed in a desired position.

Other methods to hold body tissue for treatment have been used including graspers, suction, and temporary anchors, however all these devices generally work by pulling the tissue into position. Devices disclosed herein can be configured to hold tissue in a desired position through expansion of part of or the entire device in the lumen.

Disclosed herein are devices that can be placed temporarily or permanently in a biological lumen to manipulate tissue into a desired orientation. In one embodiment, the device is an expandable member such as, for example, a stent that can be used to create a working space. The expandable member may be collapsed and removed upon the termination of the procedure. In some embodiments, the expandable member may be made of a shape memory material that is self-expanding, such as nitinol or elgiloy.

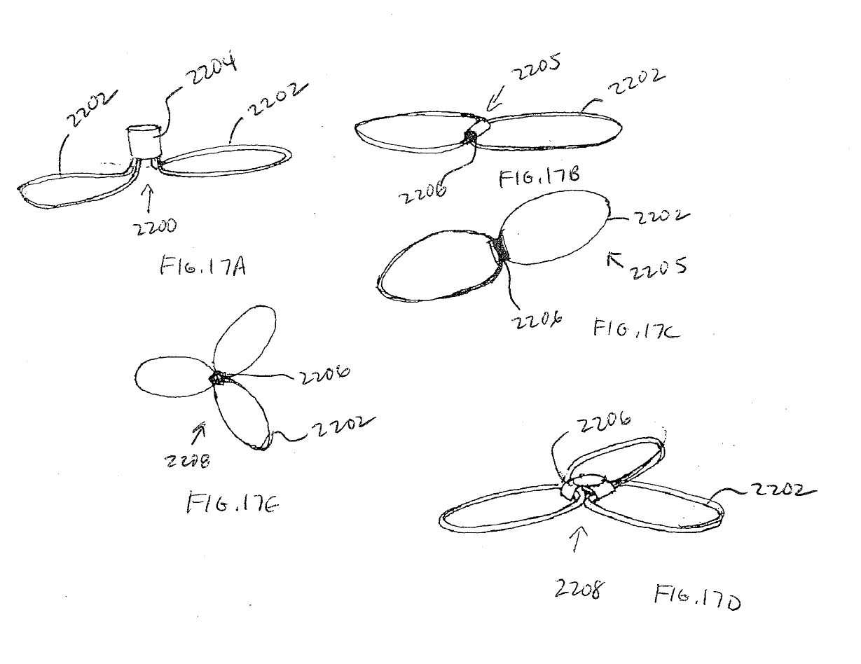

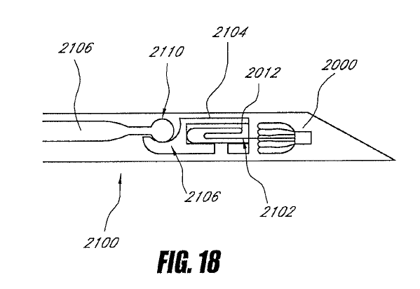

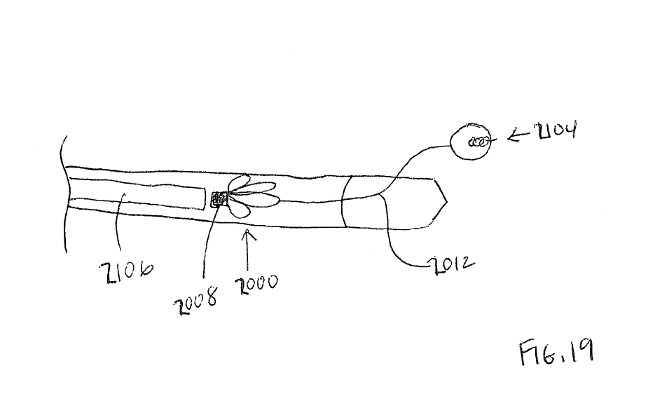

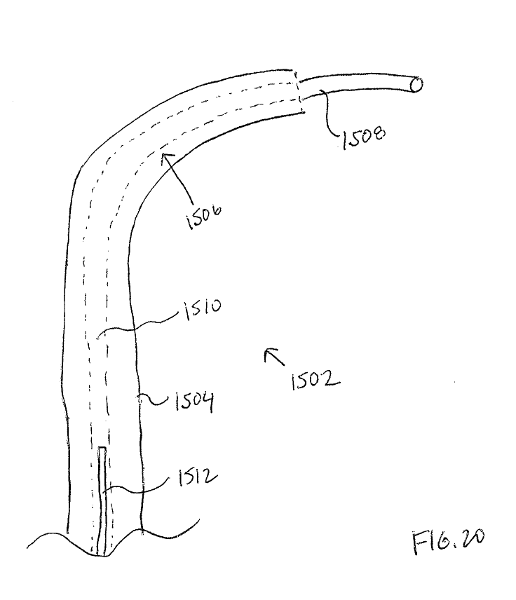

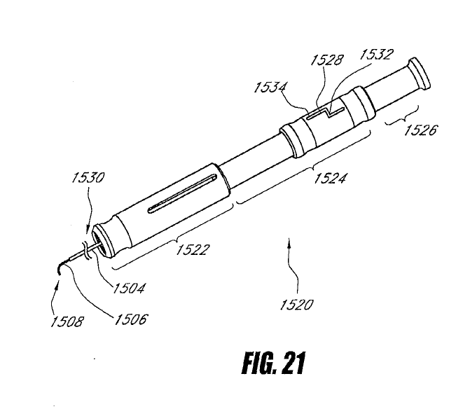

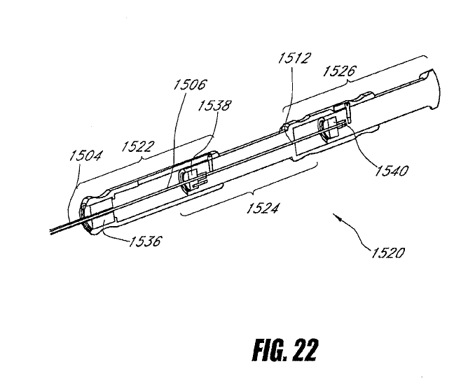

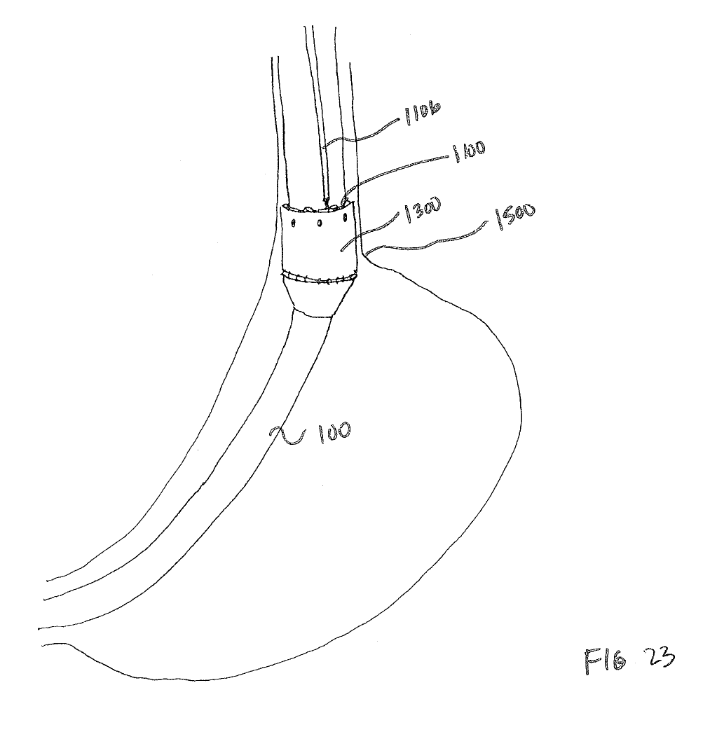

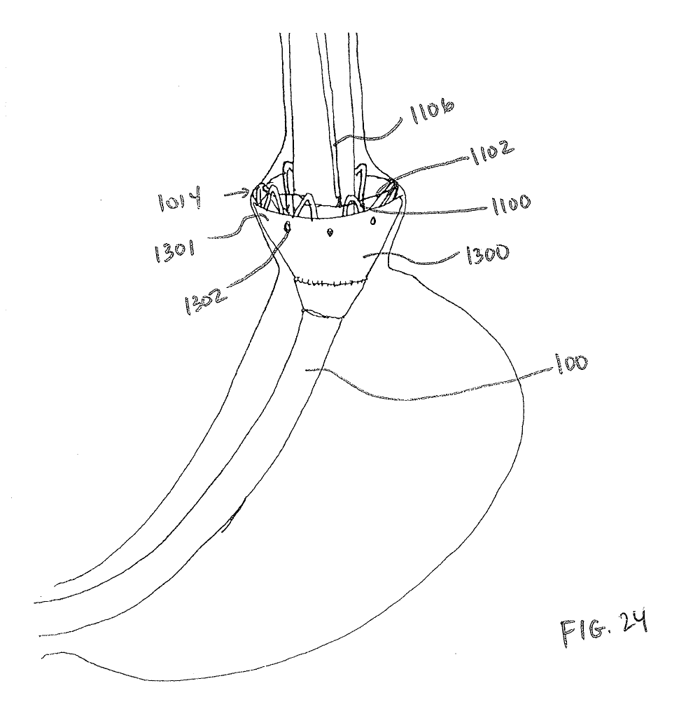

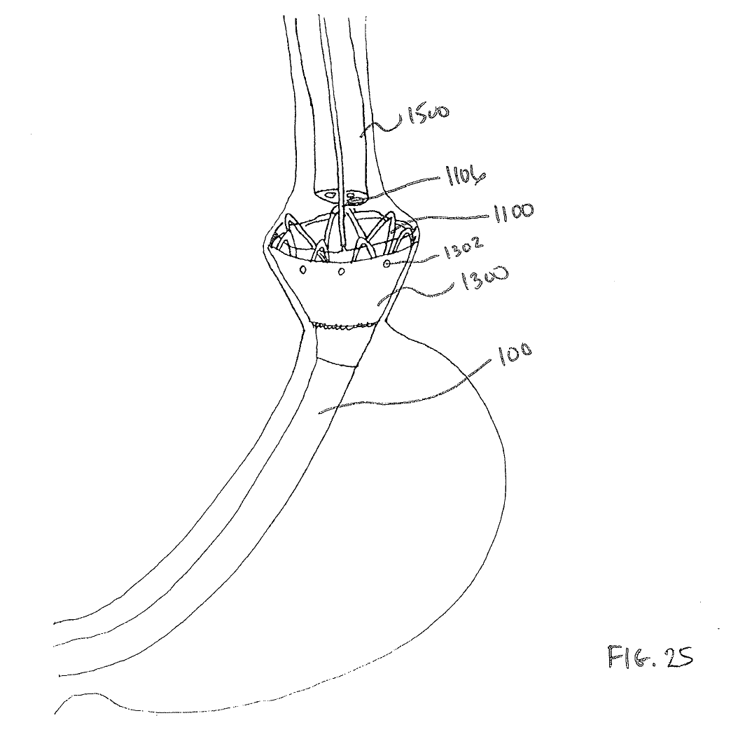

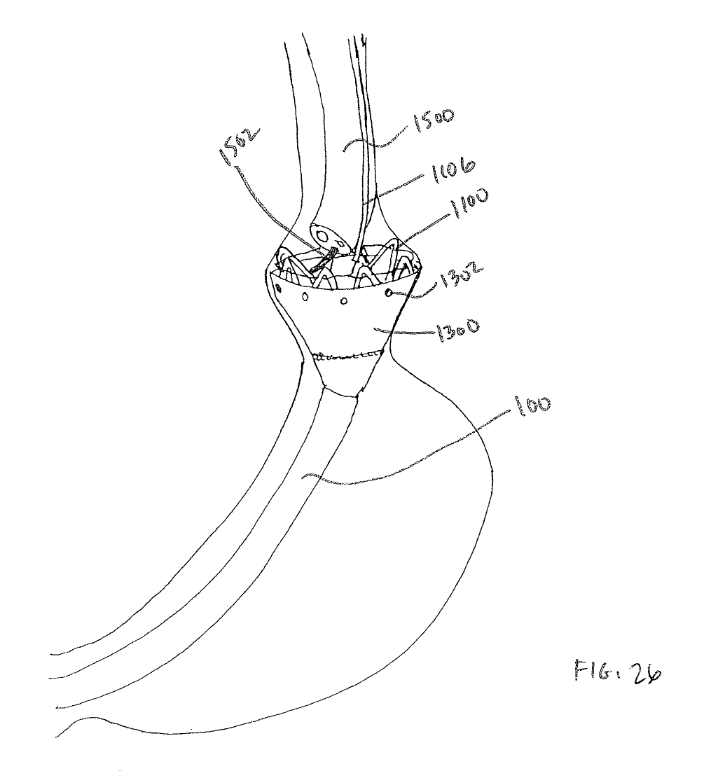

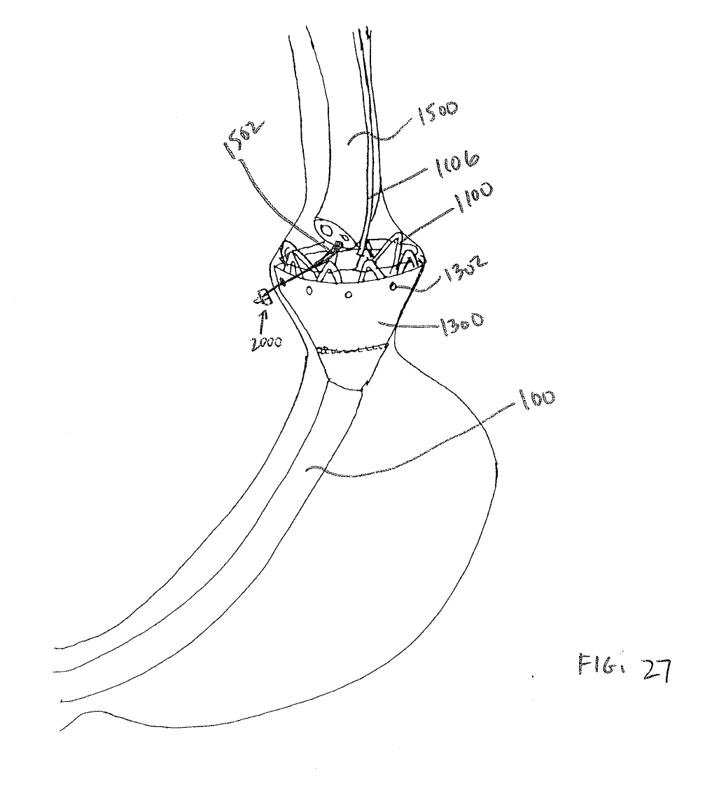

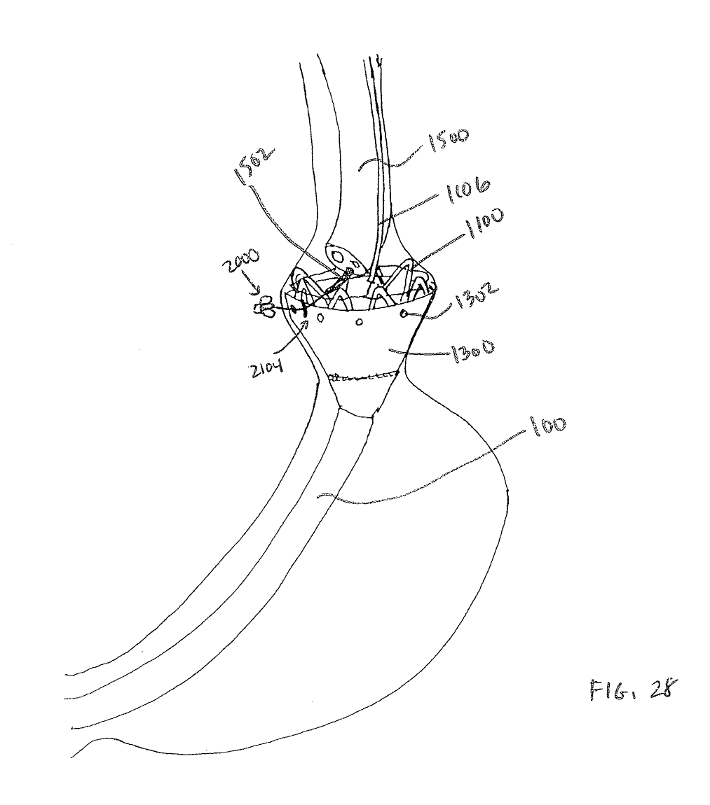

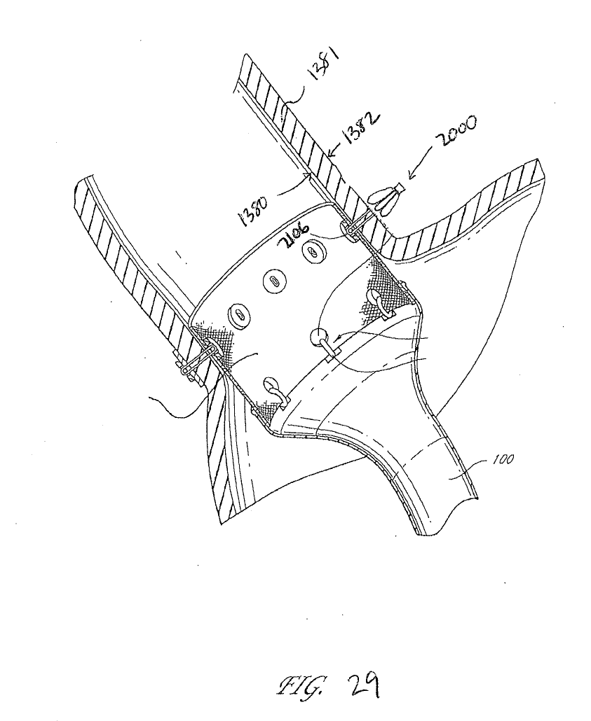

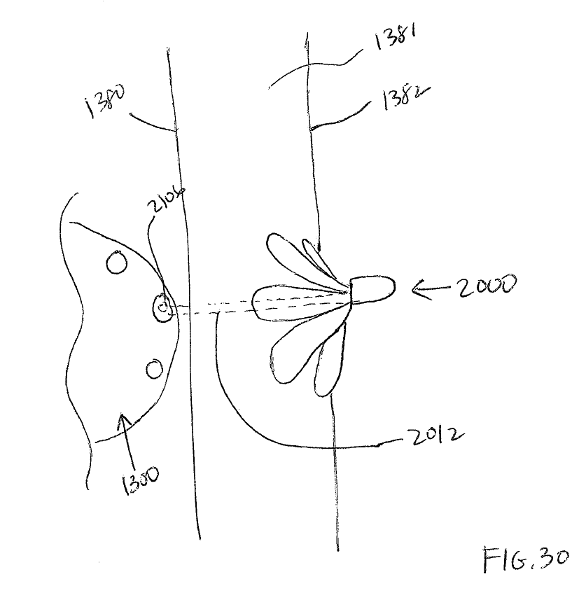

FIG. 2 illustrates an embodiment of a wire 1000 that may be used to form a stent 1100 (shown in FIGS. 3A-3C), according to one embodiment of the invention. Stent 1100 may be made of one or more wires 1000 shaped into a plurality of struts 1002 having substantially straight portions 1012 and curved portions 1004 near the apex 1014 of the stent 1000 for creating an opening through which the tissue can be accessed. The curved portions 1004 of struts 1002 can form a “bell” shape as they approach the apex 1014 to advantageously provide better access to tissue, compared to conventional Z-stents without such curved portions 1004. In some embodiments, the radius of curvature of the curved portions 1004 may be between about 0 and 180 degrees, such as about 45 to 135 degrees, or about 60 to 120 degrees in some embodiments. In some embodiments, length of curved portions 1004 of struts 1002 are at least about 20%, 30%, 40%, 50%, 60%, 70%, or more of the total length of the wire 1000. In some embodiments, wire 1000 used to form stent 1100 includes between about 4-32, such as 8-24, 12-20, or about 16 struts.

Ends 1018, 1020 of the wire may be attached, such as laser-welded, soldered, or otherwise adhered together to turn the wire form into a three-dimensional structure that has an inner lumen therethrough in some embodiments. The wire may have any appropriate diameter according to the desired clinical result. In some embodiments, the diameter of the wire is between about 0.010″ to 0.040″, between about 0.020″ and 0.030″, or about 0.026″ in other embodiments. In some embodiments, the wire 1000 is configured to create sufficient expansion force to expand the tissue of a body lumen, such as, e.g., the gastroesophageal junction. While the expandable member, e.g., stent 1100 could be laser cut in certain embodiments, it is preferred in some embodiments that the structure be formed from a wire instead to advantageously decrease the possibility of abrasion or damage to a suture interacting with the expandable member as will be described below. Furthermore, a stent 1100 formed from a wire can be less traumatic to luminal tissue and associated structures than a laser-cut stent in some embodiments.

One or more of the apex 1014 and base 1022 portions of the stent 1100 may form open loops or eyelets 1006 (at apex), 1024 (at base) as shown configured to allow the passage of a control element therethrough. In some embodiments, stent includes between about 2-16, 4-12, 6-10, or 8 apical and/or basal eyelets. While each apical 1014 and basal 1022 anchor point of the stent 1100 may include an eyelet, in some embodiments, some points may not include an eyelet, such as every other point in some embodiments. Control elements that can be actuated to collapse or expand the stent can be at different points along the distal section of the catheter. In some embodiments, a first control element, e.g., a suture loop, can control the expansion or contraction of the apex (proximal) end of the stent while a second control element can control the base (distal) end of the stent. The control elements can function in concert, or alternatively independently of each other to selectively collapse or expand the proximal and/or distal ends of the stent, respectively. In one embodiment, one of the proximal or distal ends of the stent 1100 can be maintained in a relatively expanded position while the other end of the stent is in a relatively contracted position, that is, the inside diameter of a first end of the stent is larger than the inside diameter of a second end of the stent to create a working channel, creating a funnel-like shape. The funnel can be aligned either distally or proximally with respect to the body lumen, depending on the desired clinical result. The expansion or collapsation of a portion of the stent can be locked at any position (e.g., fully expanded, fully collapsed, or at any intermediate position) by an actuating element on the control catheter, such as at the proximal end of the control catheter.

In some embodiments, at least three, four, five, or more levels of the stent, not necessarily at the proximal or distal ends, may be independently actuated (e.g., radially expanded or collapsed) using control elements.

FIG. 3A illustrates a top view of a stent 1100, such as formed from wire 1000 as illustrated in FIG. 2, with proximal end 1014 of stent 1100 in an expanded configuration according to one embodiment of the invention. As also shown in FIG. 2, stent 1100 includes struts 1002 with curved proximal portions 1004 and eyelets 1006 near the apex portion 1014 of the stent 1100. Eyelets 1024 can also be present on the base 1022 portion of the stent (not shown in FIG. 3A). Control element 1102 extends from control actuating element 1106 through eyelets 1006 at proximal end 1014 of the stent 1100 forming a loop. Control element 1102 may be a suture loop attached at knot 1104. Distal end 1022 of the stent is shown constrained within cuff as will be further described herein. In other embodiments, the control element 1102 may be a wire or other tetherable element.

In some embodiments, stent 1100 can be configured to fit at least partially within an attachment cuff 1300, and further interface with the control element 1102 of the stent 1100. The attachment cuff 1300 is elastic and compressible in some embodiments, and may be made of a fabric material in some embodiments. The attachment cuff 1300 is preferably made of a material that does not promote tissue ingrowth in some embodiments. As better illustrated in FIG. 3B, which is a side view of the stent 1100 within the attachment cuff 1300, attachment cuff 1300 can include a first plurality of apertures 1302 near its proximal end 1301 for receiving tissue anchors (also referred to herein as tissue fasteners). In some embodiments, reinforcing rings, such as, e.g., stitching or a grommet, are present around the apertures to strengthen the attachment and help prevent tissue damage when, for example, a curved needle is used to deploy the tissue fasteners, as will be described later in the application. In some embodiments, the attachment cuff 1300 can include axial reinforcing structures 1312, such as ribs, to prevent eversion of the cuff without interfering with radial expansion or collapsation of the cuff from movement of the stent or peristalsis of the lumen. The cuff 1300 can also include a second plurality of apertures 1314 or other attachment structures near its distal end 1303 for attachment to another device, which can be a gastrointestinal sleeve 100 in some embodiments.

FIG. 3C illustrates a top view of the stent 1100 of FIG. 3B in a collapsed configuration caused by actuation of control actuating element 1106 (e.g., control catheter as shown), which causes control element 1102 to partially retract into control actuating element 1106, thus contracting in length and diameter around eyelets 1006 of stent 1100, collapsing the stent as shown. FIG. 3D shows a side view of the stent 1100 shown in FIG. 3C, along with various features of the attachment cuff 1300 as previously described.

FIG. 3E illustrates a perspective view of the stent 1100 shown in FIGS. 3A-3B, with elements as previously described. The diameter D1 of the stent 1100 can be increased or decreased depending on the desired clinical result by actuation of control catheter 1106 which adjusts tension on control element 1102 running through eyelets 1006. In some embodiments, the diameter D2 of the stent 1100 can be similarly adjusted, in concert with or independently of diameter D1 via a second control element (not shown).

In one embodiment, about 2 pounds of force is required to collapse the stent 1100 completely by actuating the control handle 1222 in an appropriate direction. In other embodiments, no more than about 3, 2.5, 2, 1.75, 1.5, 1.25, 1 pound, or less of force is required to collapse the stent 1100.

In some embodiments, the stent collapses to a diameter of between about 0.15″ to 0.55″, 0.25″ to 0.45″, or about 0.35″. In some embodiments, in its fully unstressed state, the stent expands to a diameter of about 1.65″ to 2.65″, about 1.85″ to 2.45″, or about 2.15″. When opened within the esophagus, the stent will expand to between about 0.82″ and 1.2″ (20-30 mm) in some embodiments.

In other embodiments, the stent 1100 could have an unstressed non-cylindrical shape, such as a funnel or hyperboloid shape with a first radial diameter greater than the second radial diameter in its unstressed shape, and the control catheter 1106 would only need to control the end of the stent 1100 with the greater diameter when in its relaxed state, to adjust the working space of the body lumen. In some embodiments, the first radial diameter is at least about 10%, 20%, 30%, 40%, 50%, 75%, 100%, or more greater than the second radial diameter.

In some embodiments, the stent has one or more atraumatic end portions. These can be, for example, wire eyelets or loops as illustrated or have other materials covering or coating the apex of the stent bends to make them more atraumatic, such as silicone, a polymer, or the like.

In some embodiments, the space creator could have small barbs on the outer circumference of the stent, such as at eyelets, curved portions, or relatively straightened portions, for temporary attachment to the body lumen so the stent collapses the stomach down when the stent itself is collapsed. In some embodiments, screws, and/or suction devices could be incorporated into the stent so that as stent pushes against the tissue wall, it also holds the tissue wall fixed and creates counter-traction. This would enable easier passage of needles or other devices that are being passed from inside the lumen to the outside.

### Attachment Cuff

As noted above, the stent can be releasably coupled to an attachment cuff during endoscopic delivery, such as, for example, interleaving the control element with a feature such as stitching on the cuff. The attachment cuff comprises a highly flexible tubular wall extending between a proximal (superior) end and a distal (interior) end. The wall may be permeable or substantially impermeable to body fluids, and may comprise any of a variety of weave densities and/or aperture patterns either to effect flexibility, fluid transport, or to accommodate attachment as is discussed further below.

The axial length of the cuff 1300 between the proximal end 1301 and distal end 1303 can be varied considerably, depending upon the desired attachment configuration. In general, axial lengths within the range of from about 0.25 inches to about 6 inches will be used. Axial lengths within the range of from about 0.5 inches to about 2.0 inches may be sufficient to support a detachable endolumenal bypass sleeve as contemplated herein. In general, the axial length of the attachment cuff 1300 may be influenced by the desired location of the seam between the attachment cuff 1300 and the sleeve 100, or other device which is to be attached to the cuff 1300.

The attachment cuff 1300 may be constructed from any of a variety of materials which are sufficiently flexible and stable in the environment of the stomach. Suitable materials may include woven or nonwoven fibers, fabrics or extrusions using materials such as polyester velour (Dacron), polyurethane, polyamide, ePTFE, various densities of polyethylene, polyethylene terephthalate, silicone, or other materials which in the form presented exhibit sufficient compliance, stretch, strength, and stability in the gastric environment.

The inside diameter of the cuff 1300 can also be varied, depending upon the desired clinical performance. For example, the cuff 1300 may be provided with a stoma or inside diameter which is less than the inside diameter of the adjacent esophagus. Alternatively, the inside diameter of the cuff 1300 may be approximately equal to or even greater than the native esophagus. In general, inside diameters within the range of from about 15 mm to about 40 mm are contemplated, and often within the range of from about 20 mm to about 35 mm for use in human adults.

As shown in FIGS. 3B and 3D above, the cuff 1300 is provided with a plurality of attachment structures in the form of apertures 1302. These apertures 1302 are provided to facilitate anchoring of the cuff 1300 to the adjacent tissue. In either an endoscopic or surgical implantation, a plurality of tissue anchors will be pre-attached to, or advanced through the wall of the cuff 1300 and transmurally through the adjacent tissue as is discussed elsewhere herein. Provision of a plurality of anchoring points such as apertures or other structures which facilitate positioning and/or attachment of tissue anchors may desirably help with anchor location as well as reduce the amount of force necessary to advance t-tags or other anchoring structures through the wall of the cuff 1300.

In an embodiment which utilizes apertures 1302 to facilitate tissue anchoring, the number of apertures 1302 may correspond to or be greater than the total anticipated number of tissue anchors. In general, at least about four apertures 1302 and as many as eighteen or twenty are presently contemplated, with from about eight apertures to about sixteen apertures presently preferred. In one embodiment, twelve tissue anchors are used.

Preferably, the apertures 1302 in an embodiment of the cuff 1300 made from a thin walled woven or non-woven material will be provided with a reinforcement ring (one reinforcing ring per aperture, or one reinforcing ring for the implant, superior to the apertures 1302) to prevent pull-out of the associated anchoring structures, as will be appreciated by those of skill in the art in view of the disclosure herein. The reinforcement ring, where used, may be a separate component such as a grommet attached at each aperture to the cuff 1300 such as by thermal bonding, adhesives, mechanical interference or other technique. Alternatively, particularly in the case of a fabric cuff 1300, the reinforcement may be provided by stitching around the perimeter of the aperture 1302 in the manner of a buttonhole as is understood in the art.

As shown in FIG. 3B above, each of the plurality of apertures 1302 resides in a common transverse plane, positioned in the patient at or slightly above the gastroesophageal junction. Alternatively, the apertures 1302 may be provided in two or three or more transverse planes, such as to permit attachment points in a zig-zag orientation around the circumference of the attachment cuff 1300. For example, a first set of apertures (such as every other aperture) may be axially displaced from a second set of apertures by a distance within the range of from about 1 mm to about 10 mm, to provide a first and a second transverse attachment plane. Axially staggering the location of the attachment apertures may be desirable depending upon the number and configuration of tissue anchors and tissue anchor reinforcement structures as may be apparent to those of skill in the art in view of the disclosure herein.

Referring to FIG. 3B, a plurality of attachment points 1314 may also be provided on the cuff 1300, such as near the distal end 1303, for permanently or removably attaching the bypass sleeve 100 or other device. In the illustrated embodiment, the attachment points 1314 each comprise an aperture for receiving a suture hook, clip or other interference coupling, magnet assisted coupling or other link (not shown) to couple the bypass sleeve 100 to the cuff 1300. The bypass sleeve 100 may be attached to the cuff 1300 in any of a variety of ways, such as is discussed elsewhere herein. In general, the present inventors contemplate a releasable attachment between the sleeve 100 and cuff 1300, to permit removal and/or exchange of the sleeve 100 as has been discussed elsewhere herein. Further embodiments of attachment cuffs that can be used or modified for use with stents and other devices disclosed herein are described, for example, in paragraphs [0051] to [0062] and FIGS. 1-3 of U.S. Pat. Pub. No. 2007-0010866 A1 to Dann et al., which is hereby incorporated by reference in its entirety. Sleeve material and embodiments, for example, can be as described in previous disclosures, such as disclosed in the Kagan '892 publication, for example, at FIGS. 11-31 and the accompanying disclosure at, e.g., paragraphs [0241] to [0312] of the publication, or, for example, at paragraphs [0174] to [0185] of the Dann '074 publication, both of which are incorporated by reference in their entirety.

Use of an attachment cuff 1300 rather than attaching a sleeve 100 directly to the luminal wall using tissue fasteners can advantageously decouple the food transport function of the sleeve 100 from the attachment function of the cuff 1300 and allow different materials to be used for the sleeve and the cuff, depending on the desired clinical result. In some embodiments, the cuff 1300 can be at least partially radioopaque, and thus could be seen under fluoroscopy. Having a discrete cuff 1300 with different properties from a sleeve 100 can also allow for different leakage-prevention features to be present in the cuff 1300 itself in some embodiments.

### Control Catheter

FIG. 4 is a perspective view of a control catheter 1106 that utilizes a control element 1102, such as a tether loop to actuate an intraluminal space-creating device, according to one embodiment of the invention. Control catheter 1106 includes catheter housing 1120, and collet adjuster 1124 and control shaft grip 1222 proximal to and operably connected to housing 1120 as shown. Distal to and operably connected to catheter housing 1120 is introducer plug 1226 and inner 1200 and outer catheters 1202. Control catheter 1106 utilizes a loop of suture 1102 that runs around the proximal eyelets 1006 of the stent 1100. When the suture 1102 is retracted into the catheter 1106, the eyelets 1006 are pulled together, collapsing the top of the stent 1100. In some embodiments, the suture loop 1006 is made of lubricious, strong, bondable material (e.g., high density polyethelene, also known as HDPE; Teflon, GoreTex, polypropylene in other non-limiting embodiments). In some embodiments, the catheter 1106 may have a plurality of suture strands, such as two suture strands, one strand running through the proximal eyelets 1006 of the stent 1100, and one strand running through the distal eyelets 1024 of the stent 1100. The proximal eyelets 1006 and distal eyelets 1024 may be controlled independently or simultaneously.

FIG. 5 is a cross-section of control catheter 1106 through line A-A of FIG. 4, with circled areas B (FIG. 6), C (FIG. 7), D (FIG. 8) and E (FIG. 9) shown in greater detail in the respective subsequent figures.

In some embodiments, a control catheter has a proximal end and a distal end, with an elongate control element operably attached to an intermediate actuating element housed at least partially within the control catheter. The elongate control element can be attached to the intermediate actuating element at an anchoring point or aperture on the intermediate actuating element, such as at or near the distal end of the intermediate actuating element. The elongate control element extends coaxially along a longitudinal axis of the control catheter that is preferably less than the entire axial length of the control catheter in some embodiments. The intermediate actuating element can be operably connected (e.g., more proximally) to a proximal control handle. When the control handle is moved in an appropriate direction, the intermediate actuating element attached to the control handle will also move along with the elongate control element attached to the intermediate actuating element more distally. The intermediate actuating element may be, for example, a tube, such as an inner catheter member, or an elongate member such as a rod or wire in some embodiments residing at least partially within an inner catheter member. The presence of an intermediate actuating element running within the control catheter and between the proximal end of the control catheter and the elongate control element situated more distally within the control catheter can advantageously reduce friction or force that may damage the elongate control element, as opposed to a longer elongate control element that is directly connected to a proximal control handle. A shorter elongate control element also affords greater flexibility in the materials that may be used for the elongate control element. One embodiment of such a system is described in the next paragraph.

FIG. 6 is a close-up detail view of area B illustrated in FIG. 5. Shown is outer catheter 1202 slidably movable with respect to inner catheter 1200. The sutures 1102 are fixed to the inner catheter sleeve 1200 near or at its distal end. The inner catheter sleeve 1200 is movable relative to an outer catheter sleeve 1202 to pull the sutures 1102 into the control catheter 1106 and collapse the stent 1100. This configuration advantageously eliminates the need for the sutures 1102 to run all the way up to the entire axial length of the control catheter 1106 to the proximal part of the catheter 1106 (outside the body) and reduces friction on the sutures 1102, as friction from catheter 1106 movement is not transferred as much to the suture loop 1102, allowing the stent 1100 to move freely with lower risk of damaging or breaking the suture(s) 1102. Suture 1102 may be anchored to the distal end of a wire 1240 as discussed below in connection with FIG. 9. Inner catheters 1200 and outer catheters 1202 can be made of any appropriate material, such as low friction polyimide material in some embodiments. In some embodiments, the outer catheter 1202 has an outer diameter between about 0.45″ and 0.85″, such as about 0.065″, and an inner diameter of between 0.025″ and 0.065″, such as about 0.045″. In some embodiments, within the inner catheter 1200 is a rod such as a nitinol wire 1240 that allows the inner catheter 1200—outer catheter 1202 construct to bend without kinking.

FIG. 7 is a close-up detail view of area C illustrated in FIG. 5. Shown are collet 1228 and collet adjuster 1224. Collet adjuster 1224 can be threadably connected to collet 1228 and rotation of the collet adjuster 1224 in an appropriate direction will lock the inner catheter 1200 and outer catheter 1202 in a desired axial position. Also shown is wire 1240 within inner catheter as previously described.

In some embodiments, an elongate element configured to be placed within a body lumen, such as a catheter or wire having a radial diameter includes a slidable distal plug configured to be attached to an end of a device, such as, for example, a larger diameter catheter, sleeve, tube, or introducer also configured to be placed within a body lumen having a radial diameter greater than the radial diameter of the elongate element, such as at least about a 1.5×, 2×, 3×, 4×, 5×, or more times greater radial diameter relative to the elongate element. As will be discussed further below, when not in use the plug can be secured to a distal portion of the elongate element and removed from the body lumen, leaving the larger diameter device in place. The sliding plug advantageously reduces or eliminates the risk that body lumen tissue pinches between the smaller diameter elongate element and the larger device placed coaxially over the elongate element when deployed within the body lumen.

FIG. 8 is a close-up detail view of area D illustrated in FIG. 5. In some embodiments as shown, the control catheter 1106 includes a distal plug 1226 that may be made of an appropriate material, such as molded silicone, that slides along the outer diameter of the outer catheter 1202. The plug 1226 can be configured to mate with an overtube 1400 introducer tip 1402, as shown in FIG. 10A. If an overtube 1400 is being placed over the control catheter 1106, the plug 1226 is mated with the introducer tip 1402 to eliminate the possibility for tissue to pinch between the catheter 1106 and the introducer tip 1402. When the plug 1226 is disconnected from the overtube 1400, as shown in FIG. 10B and not in use, it can be secured to the distal part of the handle mechanism 1220 of control catheter 1106 as shown in FIG. 10C.

FIG. 9 is a close-up detail view of area E illustrated in FIG. 5. Shown are the inner catheter 1200 and outer catheter 1202, as well as the wire 1240 with an aperture 1242 near the distal end of the wire 1240 for attachment of a control element 1102 such as a suture. In this way, suture 1102 can be actuated remotely by movement of proximal control shaft grip 1222 connected to proximal end of wire 1240 even though suture is attached to wire 1240 more distally. In some embodiments, distance from aperture 1242 configured to serve as a proximal anchor point for suture 1102 to the distal end of control catheter 1106 is no more than about 70%, 60%, 50%, 40%, 30%, 20%, 10%, or less of the total axial length of the control catheter. As noted above, this can advantageously eliminates the need for the sutures 1102 to run all the way up to the proximal part of the catheter 1106 (outside the body) and reduces friction on the sutures 1102, as friction from catheter 1106 movement is not transferred as much to the suture loop 1102, allowing the stent 1100 to move freely with lower risk of damaging or breaking the sutures 1102.

In some embodiments, the aperture 1242 or control element anchoring point to control catheter 1106 may be at or near the distal end of the inner catheter 1200. The outer catheter 1202 has a rounded tip 1206 in some embodiments where the suture 1102 exits to keep the suture 1102 from becoming weakened or frayed as it pulls in or out of the catheter 1106.

FIG. 11 illustrates a cut-away schematic view of an embodiment of a control catheter with a plurality of control elements. As shown, the control catheter 1150 includes a telescoping inner catheter 1200 and outer catheter 1202 that may be as previously described. Control elements as shown can be a plurality of sutures: proximal sutures 1152, 1154 and distal sutures 1156, 1158 each forming a loop at their distal ends. The distal ends of the suture loops 1152, 1154, 1156, 1158 extend through the lumen of inner catheter 1200 and the proximal ends of the suture loops 1152, 1154, 1156, 1158 are attached proximally to the inner catheter 1200, such as at distal anchor point 1162, which may be an aperture in some embodiments. In other embodiments, instead of being attached to the inner catheter 1200, some or all suture loops may be attached at or near the distal end of a wire or rod residing within inner catheter 1200 as previously described. Distal suture loop 1156 can be threaded through distal eyelets of a stent (such as, for example, a stent illustrated in FIG. 3C) as well as around a cuff (as shown, for example, in FIG. 3A). Distal suture loop 1158 is threaded through distal eyelets of a stent but not a cuff in some embodiments. Similarly, proximal suture loop 1152 may exit the outer catheter 1202 through a first aperture or notch 1160 in the outer catheter 1202 and is threaded through the proximal end of the cuff and the stent, and then back through another aperture or notch 1161 in the outer catheter 1202. Suture loop 1154 can be looped similarly to 1152 around eyelets of a stent but without circumscribing the cuff. If the inner catheter 1200 is pulled proximally relative to outer catheter 1202 the loops 1152, 1154, 1156, 1158 can close together, collapsing the stent and the cuff and allowing for some repositioning of the cuff-stent assembly. In such an embodiment, the suture loops 1152, 1154, 1156, 1158 do not move axially relative to the inner catheter 1200 to minimize or eliminate friction between the tails of the suture loops 1152, 1154, 1156, 1158 and the lumen of the inner catheter 1200. To release and remove the expanded stent, loops 1152, 1156 may be cut or pulled through the catheter, releasing the stent from the cuff. Next, to complete the removal process, the inner catheter 1200 may be pulled proximally relative to the outer catheter 1202, collapsing the stent and allowing the entire stent-catheter assembly to be removed, such as through an overtube. In some embodiments, loops 1152 and 1156 that may be threaded through both stent and cuff need not be present. In some embodiments, proximal loop(s) 1152, 1154 and distal loop(s) 1156, 1158 may be controlled independently on each other, for example, if proximal loops were attached to a first catheter having a first control handle and distal loops were attached to a second catheter coaxial with the first catheter and controlled by a second control handle.

### Other Applications

The stent-based space-creating device could be used in other access points in the GI tract or other tube-like structures where space needs to be maintained. For example transbiliary, transrectal, transvaginal, transcolonic, transintestinal, or other procedures could be performed by deploying the stent in these structures, passing through the tissue wall (as described above) and then removing the stent once the incision is closed. For example, the space-creating device may be used for improved visualization during diagnostic or therapeutic upper GI endoscopy or colonoscopy procedures.

In some embodiments, the space creator could have small barbs on the outer circumference of the stent, such as at eyelets, curved portions, or relatively straightened portions, for temporary attachment to the body lumen so the stent collapses the stomach down when the stent itself is collapsed.

Barbs, screws, or suction devices could be incorporated into the stent so that as stent pushes against the tissue wall, it also holds the tissue wall fixed and creates counter-traction. This would enable easier passage of needles or other devices that are being passed from inside the lumen to the outside.

The space-creating device, in other embodiments, could be one or more inflatable structures, such as balloons, which can be temporarily inflated to open up a lumen in a desired manner to create a working space. In some embodiments, the space-creating device could be an expandable braided mesh sphere or tube made from a shape memory material such as nitinol.

In other embodiments, the space-creating device may be the expandable flanges of an overtube, or other similar configuration, e.g., as described in paragraph [0273] of U.S. Pat. Pub. No. 2007/0198074 A1, hereby incorporated by reference in its entirety.

### Expandable Fastener System

Also disclosed herein is a fastening system that can be used, for example, to anchor a device to one or more tissue walls using at least a first retention element and a second retention element operably connected by a tension element. A first retention element, in some embodiments, includes a plurality of elongate structures shaped into a plurality of petals, the petals operably connected to a central hub. The plurality of petals can form a proximally facing surface which rests against a tissue surface, such as a serosal surface to retain the device. The actual footprint of the retention element, that is, the surface area of the elongate structures that form the plurality of petals resting against the tissue surface is preferably substantially less than the effective footprint of the retention element, as will be described further below. Not to be limited by theory, a fastening system could be designed to minimize adverse tissue reactions caused by less of a surface area of the retention element exerting pressure on the tissue surface while at the same time maximizing the retention efficacy of the retention element.

FIG. 12A illustrates an embodiment of a first retention element 2000, which can be a distal retention element, that is configured to rest against a first surface, such as the serosal surface of a tissue wall. Retention element 2000 includes a plurality of retention surfaces 2002 as shown, which may be elongate structures such as, for example, wires, shaped into a plurality of petals 2006 as shown. The retention surfaces may be made of any appropriate material, such as nitinol, elgiloy, shape memory polymers, or stainless steel in some embodiments, and can be configured such that the retention element can advantageously be transformed from a first, low-profile reduced configuration during delivery to a second, expanded configuration while in use, and if necessary, back to the first low-profile reduced configuration if the retention element is later removed from the tissue. In some embodiments, the wire has a thickness of between about 0.001 inch and 0.05 inches, such as between about 0.005 and 0.010 inches, and about 0.006 inches in certain embodiments. Each wire, in some embodiments, has a running length of between about 0.1 inches to 1.5 inches, such as between about 0.30 inches and 1 inches, and between about 0.50 inches and 0.90 inches in some embodiments. At least a portion of the retention element 2000 is radioopaque in certain embodiments.

Retention element 2000 can have any number of petals 2006 depending on the desired clinical result. In some embodiments, a retention element 2000 includes at least about 2 but no more than about 20 petals, such as at least about 3 petals but no more than about 12 petals, such as 4, 5, 6, 7, 8, 9, 10, 11, or 12 petals in some embodiments, and 6 petals as shown in FIG. 1. Petals may be uniformly or substantially uniformly spaced apart as shown in FIG. 1, illustrating 6 petals each spaced apart by 60 degrees, or irregularly spaced apart in other embodiments.

Petals 2006 may be of any desired shape, but preferably lack sharp edges in some embodiments to reduce the risk of inadvertent puncturing or damage to the tissue. In some embodiments, the distal portion 2009 of each petal 2006 has a semi-circular shape to advantageously increase the effective surface area of the tissue to be retained (described in greater detail below), although other curved and non-curved shapes are also within the scope of the invention.

In some embodiments, the petals 2006 of the distal retention element 2000 are made of a relatively compliant material, that is, the petals 2006 will reversibly deform when a proximal force is exerted on a tension element operably connected to the distal end of the retention element (described further below) to prevent damage to tissue of which the distal retention element 2000 bears upon and/or the retention element 2000 itself. In some embodiments, the petals 2006 are made of a material and configuration to produce a compliance of at least about 0.05, 0.1, 0.2, 0.3, 0.4, 0.5, 0.75, 1, 1.25, 1.5, 1.75, 2, 2.5, 3 pounds, or more, such as at least about 1.5 pounds in some embodiments. In some embodiments, retention elements 2000 can include force-sensing elements, such as, for example, a cantilever and a sensor/transducer, which can be operably connected to a data collection/transmission device to record the amount of force exerted on the retention element 2000.

A spring constant relates the force exerted by a spring to the distance it is stretched by a spring constant, k, measured in force per length, F=kx. The retention element 2000 may be configured to have a specific spring constant depending on the desired clinical result. In some embodiments, the spring constant of a retention element 2000 may be between about 1-5 pounds per inch, such as between about 2-4 pounds per inch, between 2.5-3.5 pounds per inch, 2.75-3.25 pounds per inch, or about 3 pounds per inch in some embodiments. In some embodiments, the spring constant may be at least about 0.3, 0.5, 0.7, 1, 1.5, 2, 2.5, 3, 3.5, 4, 4.5, 5, or more pounds per square inch, or no more than about 5, 4.5, 4, 3.5, 3, 2.5, 2, 1, 0.7, 0.5, 0.3, or less pounds per square inch in other embodiments.

In some embodiments, petals 2006 can be coated with one or more materials depending on the desired clinical result, such as, for example, to increase fibrosis and thus potentially increase the retention capability of the distal retention element 2000, or to prevent growth of a pathogen on the retention element 2000. In some embodiments, the petals 2006 can be coated with a tissue-ingrowth material. The tissue-ingrowth material can be e-PTFE, Gore Dual Mesh, Bard Dulex, or Dermagraft. The material may also be a tissue graft material, such as small intestinal submucosa, collagen, and the like. The coating may also include a drug, such as an antibiotic, an anti-inflammatory or an anti-proliferative agent, or a growth factor, for example. In some embodiments, at least a portion of the retention element 2000 is coated with a silver compound, which has anti-microbial properties.

Petals 2006 can be operably connected to a central hub 2008 via welding, crimping, adhering, frictional force, or other means as known in the art, such as at one or both ends of the elongate structures of retention surfaces 2002. Hub 2008 may be axially in-line with a plane of the petals 2006, or can project distally from the serosal surface a certain distance, such as no more than about 20 mm, 10 mm, 5 mm, or less in some embodiments to advantageously reduce pressure around the transmural axial aperture through the tissue created by the tension element 2012.

The petal 2006 configuration allows the retention element to provide a relatively large effective “footprint” while maintaining a relatively small actual tissue-device contact area. In other words, the retention element 2000 is able to effectively retain a relatively large surface area of tissue, for attaching a device on the other side of the tissue wall, while only a relatively small surface area of the wires 2002 of the petals 2006 actually engages the tissue. Not to be limited by theory, a relatively small actual surface area that actually contacts tissue for the distal retention element 2000 could reduce the risk of a foreign body tissue reaction that may lead to undesired pressure ulceration leading to migration or failure of the tension element, infection, and/or overgrowth of fibrous tissue on the distal, e.g., serosal surface. In some embodiments, the effective footprint of the retention element 2000 is defined as the area of the smallest diameter circle 300 that still circumscribes all of the petals 2006 of the retention element 2000, as illustrated in FIG. 12B. The maximal surface area of the petals 2006 that could engage the tissue can be calculated as a function of the diameter of the wires 2002 that form each petal 2006, the running length of each wire 2002, and the total number of petals 2006. For example, in an embodiment with 6 petals with 1 wire comprising a petal 2006, a wire 2002 diameter of 0.006 inches, and a wire 2002 running length of 0.66 inches, the surface area of the wire 2002 is about 0.024 square inches (6 petals×1 wire per petal×0.006 inch wire diameter×0.66 inch wire running length). Assuming the diameter 302 (shown as a dashed line) of an outer boundary of the retention element 2000, which can be, e.g., the smallest diameter circle 300 circumscribing all of the petals 2006 is 0.30 inches, the area of the circle 300 defining the effective footprint of the retention element 2000 is 0.071 square inches. Therefore, the open space area, defined as the area of the effective footprint minus the maximal surface area of the petals 2006 is 0.47 square inches. The open space area of 0.47 square inches is thus about 66% of the total effective footprint of the retention element 2000, 0.71 square inches (thus, the surface area of the petals 2006 is about 34% of the area of total effective footprint of the retention element 2000). As noted above, the number of petals 2006, number of wires 2002 forming each petal 2006, and the running length of each wire 2002 can be varied in different embodiments depending on the desired clinical result. In some embodiments, the open space area is at least about 10%, 15%, 20%, 25%, 30%, 45%, 40%, 45%, 50%, 55%, 60%, 65%, 70%, 75%, 80%, 85%, 90% or more of the area of the effective footprint of the retention element 2000 as defined above. In some embodiments, the total surface area of the petals 2006 is no more than about 90%, 85%, 80%, 75%, 70%, 65%, 60%, 55%, 50%, 45%, 40%, 35%, 30%, 25%, 20%, 15%, 10% or less of the area of the effective footprint of the retention element 2000. In some embodiments, the diameter of the smallest diameter circle 300 circumscribing all of the petals 2006 is between about 0.05 inches to 0.70 inches, such as between about 0.10 inches to 0.50 inches, or between about 0.20 inches to 0.40 inches. In some embodiments, the total surface area of the petals 2006 is between about 0.10 square inches to 1 square inch, such as between about 0.20 square inches to 0.80 square inches, or between about 0.40 inches to 0.60 inches.

While petals 2006 of a first retention element 2000 collectively serve to bear against the tissue to retain a device operably connected by the tension element 2012 to a second retention element, each individual petal 2006 advantageously functions and is movable independently of one another to assist with load sharing. In this manner, dysfunction or failure of one or more of the petals 2006 will not affect the retention capabilities of the remaining functional petals 2006.

FIG. 13 is a transverse sectional view through line A-A of the retention element 2000 of FIG. 12A. Shown are the petals 2006 and hub 2008 which has a central lumen 2010 configured to receive a tension element 2012 therethrough. Tension element 2012 has a proximal end (not shown), distal end 2013, and elongate body 2011. Distal end 2013 of the tension element 2012 may have an enlarged portion such as a knot to secure the distal end 2013 of the tension element within a corresponding recess within central lumen 2010 of the hub 2008 via press-fitting, adhesive attachment, or other means known in the art. A second more proximal stop surface, such as another knot can be present just proximal to the opening of the central lumen 2010 of the hub 2008, and preferably has a diameter greater than that of the hub lumen to secure the tension element 2012. Tension element 2012 can be a suture in some embodiments, such as 3-0 monofilament polyprolene with a diameter of about 0.011″ in some embodiments. In other embodiments, the tension element may be made of a wire, such as, for example, nitinol, elgiloy, or stainless steel, which may be advantageous as the wire can generally be configured with a diameter smaller than that of a suture, which may decrease the likelihood that bacterial will migrate through the transmural tissue track of which tension element 1012 resides. In some embodiments, the diameter of the tension element is between about 0.001″ and 0.05″, such as between about 0.005″ and 0.02″ in some embodiments. The length of the tension element, in some embodiments, is preferably between about 100-300%, between about 100-200%, or about 150% of the thickness of the transmural tissue wall in which the tension element passes through. In some embodiments, the tension element is at least about 100%, 125%, 150%, or more of the thickness of the transmural tissue wall, or no more than about 300%, 200%, 150%, or 125% in other embodiments. The transmural tissue wall may be a stomach, esophageal, or intestinal wall in some embodiments.

Referring to FIG. 12A, each petal 2006 is formed into an inclined portion 200 and a tissue contact portion 202, which may be separated by a bend 204. The inclined portion 200 extends proximally from the hub 2008 at an angle within the range of from about 25° to about 65° with respect to the longitudinal axis of the tension element 2012. In one embodiment, the angle between the inclined portion 200 and the longitudinal axis of tension element 2012 is within the range of from about 35° to about 45° in its unstressed configuration.

The inclined portion 200 is configured to produce an axial depth 206 between the hub 2008 and the contact portion 202 of the petal 2006 which may be within the range of from about 0.1 inches to about 0.2 inches. The contact portion 202 of the petal 2006 has a length 208 measured in the radial direction within the range of from about 0.040 to about 0.100 inches.

Referring to FIG. 12A, in some embodiments, the consequence of the foregoing geometry is to produce a footprint against the tissue in which the contact portion 202 of each petal 2006 resides generally within a contact zone 210. Contact zone 210 is radially symmetrically disposed about the axis of the tension element 2012, but spaced radially outwardly from the axis as will be discussed. Contact zone 210 thus includes a width 212, between an outer boundary 214 and an inner boundary 216. The width 212 of the contact zone 210 corresponds approximately to the length 208 of the contact portion 202 of petal 2006.

The width 212 of the contact zone 210, angles of the inclined portion 200 and other dimensions may vary from embodiment to embodiment, depending upon the desired clinical result. In addition, the dimensions may be varied in use, depending upon the compressibility of the tissue to which the fastener is applied and the amount of proximal tension placed on tension element 2012. In addition, the width 212 of the contact zone 210 may change over time following implantation, as adjacent tissue remodels or other tissue responses occur. In general, however, one consequence of the foregoing geometry is to provide a central zone 218 which is free or substantially free of contact between the tissue and the retention element 2000. This allows the tissue contact zone 210 to be spaced apart from the injury site where the tension element 212 extends through the tissue, which may inhibit bacterial transport between the tissue tract and the wire-tissue contact area. Force is also distributed over a relatively large area, spaced apart from the tissue tract. Even if there is some contact between tissue and the device near the tension element injury site, pressure on the injury site is minimized due to the force distribution accomplished by the present design. This may reduce the risk of pressure necrosis of the injury site. In addition, this configuration allows a dampening of forces as tension is applied to tension element 2012 and inclined portion 200 acts as a spring biased lever arm. In an embodiment intended for transmural placement against the serosa at the gastroesophageal junction, the diameter 220 of the central zone 218 is generally at least about 0.1 inches, and may be at least about 0.15 inches, or 0.20 inches, or greater.

In one embodiment of the fastener, a six petal configuration as shown in FIG. 12A is constructed from a 0.006 inch diameter wire. Each end of each flower petal is bent into a radius of approximately 0.027 inches, such that the length of wire of each petal within the tissue contact zone 210 is approximately 0.18 inches. In a six petal embodiment, the running wire length having contact within the tissue contact zone 210 is approximately 1.13 inches, so that the area of wire surface presented to the tissue (assuming slight embedding of the wire into the tissue) is approximately 0.0068 square inches.

The diameter of the outer boundary 214 is approximately 0.300 inches, and the diameter of the inner boundary 216 is approximately 0.240 inches in the foregoing embodiment. Thus, the area of the tissue contact zone 210 is approximately 0.0255 square inches. Thus, the area of contact between the wire and the tissue is approximately 26.6% of the total area of the tissue contact zone 210, which is spaced apart from the injury site of the tension element 1012 by a distance of about 0.12 inches. In this embodiment, the width 212 of the contact zone 210 is less than half of the diameter 220 of central zone 218, or could be less than 45%, 40%, 35%, 30%, 25%, or less in certain other embodiments.

FIG. 14 is a close-up view of circled area B of FIG. 13, illustrating tension element 2012 with surfaces 2015 and 2017 to secure the tension element 2012 with respect to the hub 2008. Also shown are proximal regions of petals 2006, which are connected to the hub 2008 as described above.

FIG. 15 is a perspective view of a retention element 2020 similar to retention element 2000 illustrated in FIG. 12A, illustrating a plurality of petals 2006 operably connected to distal hub 2008, which in turn is operably connected to tension element 2012.

FIG. 17A is a perspective view of one embodiment of a retention element 2200 with two petals 2202 operably connected to a central hub 2204. FIG. 17B is a retention element 2205 similar to the retention element 2200 of FIG. 17A, with a lower profile hub 2206 that may be axially in-line or substantially axially in-line with a plane of the long axis of the petals, as noted previously. FIG. 17C is a top view of the retention element 2200 of FIG. 17B. FIG. 17D is a perspective view of an embodiment of a retention element 2208 that includes three petals 2202, with a lower profile hub 2206 as previously noted. FIG. 17E is a top view of the retention element 2208 of FIG. 17D.

A perspective view of a fastener system 2020 including a proximal retention element and a distal retention element is shown in FIG. 16A. Shown is the distal retention element 2000 with a plurality of petals 2006 connected to hub 2008, and tension element 2012 as previously described. Also shown is a proximal retention element 2104, which can be a button-shaped element secured to the tension element by knots 2194 as shown. The proximal retention element 2104 may be any of a wide variety of fasteners, such as T-tags, T-pledgets, or other fasteners, for example, those described in FIGS. 2 and 5A-7B and paragraphs [0126] to [0129] and [0136] to [0157] of U.S. Patent Pub. No. 2007/0198074 A1 to Dann et al., previously incorporated by reference in its entirety. Additional fasteners that can be used are described, for example, in U.S. Provisional Application No. 61/033,385 filed Mar. 3, 2008 and incorporated by reference in its entirety, such as, for example, in FIGS. 1-5 and the accompanying text at paragraphs [0002] to [0022] of the '385 provisional application. The proximal retention element could also be the same as the distal retention element described in connection with FIGS. 12A-15 or 17A-D, for example. FIG. 16B is another perspective view of the fastener system 2020 illustrated in FIG. 16A. FIG. 16C is an end view of the proximal retention element 2000 similar to as shown in the embodiment of FIG. 12.

### Delivery System

Systems and methods for deploying a fastener system including retention element 2000 and tension element 2012 will now be described.

FIG. 18 illustrates an embodiment of a fastener system, housed within a delivery cannula 2100, such as the curved needle driver described above. Illustrated is the distal “flower petal” retention element 2000. At least a portion of the tension element 2012, which may be a suture as shown, wire as described above, or other tether, may reside in a groove or slot 2102 within a portion of the proximal retention element 2104 configured to house the suture 2012 in place during deployment to advantageously prevent undesired damage, tangling, knotting, or the like to the tension element. The proximal retention element 2104 is in turn shown adjacent to a stylet 2106 for pushing the distal retention element 2000 and/or proximal retention element 2104 out of the delivery cannula 2100 at the appropriate time. In some embodiments as shown, the proximal retention element 2104 includes a stylet groove 2108 or other surface to reversibly couple the proximal retention element 2104 together with a complementary surface 2110 of the stylet 2106 while the proximal retention element 2104 is within the delivery cannula 2100. This configuration can help to prevent the proximal retention element 2104 from prematurely deploying together with the distal retention element 2000 on the serosal side of the tissue wall. Other means to reversibly couple the proximal retention element 2104 to the stylet 2106 can also be employed as known in the art, for example, magnets, chemical (e.g., electrolytic attachment), a weak adhesive, a releasable clamp, and the like.

In other embodiments where the proximal retention element 2104, such as a button-shaped element, is too large to fit within the delivery cannula 2100 as illustrated schematically in FIG. 19, the proximal retention element can “hang”, connected to the proximal end of the tension element 2012, outside of the delivery cannula 2100. This can ensure that the proximal retention element 2104 remains on the proximal (e.g., mucosal) side of the tissue to be cannulated. In such embodiments, the distal retention element 2000 may be loaded in reverse (that is, petals closest to the distal end of the cannula 2100, to be ejected before the hub 2008 end) into the delivery cannula 2100 as shown in FIG. 19 due to constraints on the length of the tension element 2012 due to the proximal retention element 2104 residing outside of the delivery cannula 2100. The distal retention element 2000 can “flip” 180 degrees longitudinally upon deployment across the serosal side of the tissue either by itself, or with laparoscopic assistance.

### Endoscopic Curved Needle Driver

Most endoscopes, including many enteroscopes, colonoscopes, etc, have visual imaging capabilities and one or more working channels. The working channel(s) and the line of sight of the visual imaging element are often along nearly parallel axes and these axes are only at most a few millimeters apart. Targeting the side of a lumen in the GI tract is a common need in endoscopic procedures. Often there is the need to biopsy tissue, remove polyps, apply heat or energy to an area of tissue, cannulate a duct, etc. Because of the proximity of these two axes and their parallel paths, when a tool is advanced down the working channel and into the field of view of the optics, it can be challenging to view how the end of the tool is interacting with a target (e.g., at a side of the lumen), its orientation, and how much length of the tool is outside of the scope. Some of this difficulty can be caused by the shaft of the tool obscuring the tip of the tool and some of the challenge is due to the orientation of the axes. In addition, although the tip of most scopes are steerable, it can be challenging to view and target the wall of lumen, especially if the lumen is not much bigger than the diameter of the scope.

One type of scope, an ERCP scope, is a side-viewing scope designed specifically for ERCP (endoscopic retrograde cholangiopancreatography) procedures and has a side oriented view and working channel. While this helps viewing the wall of a lumen, for example, it can generally have the same inherent issues of front viewing scopes where the working channel is near parallel and close to the axis of the line of sight. The ERCP scope tries to overcome the limitation of the working channel orientation by providing an “elevator” that allows an operator to change the angle of the instrument relative to the scope channel. Actuation is generally accomplished with guidewires and other highly flexible devices. Instruments that need stiffness, such as needle drivers, generally would not work properly with this sort of elevator mechanism.

Endoscopic tools that are deflectable with the use of guidewires and/or robotic controls have been previously described. The tools described here have a preset curved distal end section that makes the distal section of the tool form an arc as it leaves the end of the working channel in an endoscope. Advantages of this design which arc the end of the tool away from the long axis of the tool include: a more direct view of the tip of the tool; easier view on how it is interacting with a target; easier estimation of how much length of the tool is out of the working channel of the scope; and easier ability to target an area away from the main axis of the end of a scope, e.g. on the side of a lumen in the GI tract.

### End Effectors for Curved Needle Driver

The end effector of the tool can be any tool that is used in endoscopic procedures. While the end effector will primarily be described in terms of a needle driver end-effector below, other end effectors, such as graspers, cutters, snares, biopsy needles, RF electrodes, and the like can also be used with the present invention.

In some embodiments, the tool preferably includes a needle driver, and preferably has a distal section made of, for example, a shape memory material that when unconstrained forms an arc. Nitinol, elgiloy, stainless, a shape memory polymer, plastic, and the like could be used depending on the requirements of the tool. Most preferably, the tool is configured such that there is a low enough spring force to allow easy movement proximal and distal in the working channel.

The ability to torque the proximal end of the tool and cause corresponding movement of the distal end is very preferable to facilitate accurate movement of the distal end of the tool and target desired locations. In some embodiments, a hypotube, such as one made of nitinol, could be used in the shaft for better torsional rigidity. Also, supplemental supports along the shaft or radial support structure could also help increase torsional rigidity. In some embodiments the shaft of the tool can be a larger diameter than the curved section and/or the end effector. This allows improved torsional rigidity of the shaft but does not necessitate a larger end effector than is desired.

In the example of the curved needle driver, it may be desirable to use the smallest gauge needle possible to deliver a t-tag to minimize tissue trauma. In one embodiment, the shaft could have a diameter of no more than about 14 gauge, 16 gauge, 18 gauge, or less while the distal curved section has a diameter of no more than about 16 gauge, 17 gauge, 18 gauge, 19 gauge, 20 gauge, or less. The distal tip may have the same diameter of the curved section, or even smaller, such as no more than about 19 gauge, 20 gauge, 21 gauge, 22 gauge, 23 gauge, 24 gauge, or less.

### Endoscope Bracing Element

When the curved distal section of the tool is in the working channel of the endoscope, the endoscope's structure provides the force necessary to keep the distal section from assuming its curved configuration. Most preferably, the spring rate of the curved portion is low enough that this force is not sufficient to deflect any portion of the endoscope, move the tip of the endoscope when the curved section is advanced or retracted or cause any undue wear or damage to the endoscope.

However, in some embodiments, one or more supplemental bracing elements can be used with the curved tool to take some of the straightening load away from the endoscope. Ideally, these constructs would not be so stiff to take away the steering capabilities of the endoscope. In some embodiments, one possible bracing element includes an external collar on the distal end of the endoscope that stiffens a distal length of the endoscope. In another embodiment, if there is more than one working channel in the endoscope present, a stiffening element can be advanced down a working channel not occupied by the curved tool to increase the rigidity of the endoscope. A hollow stiffening element could be inserted in the tip of the working channel the curved tool will be used in to stiffen the tip of the endoscope. In such an embodiment, the stiffening element tube's inner diameter should be large enough for the tool to move through it and the proximal rim of the stiffening element needs to be tapered from ID to OD so there is no rim to catch the tool on when it is advanced into the stiffening element. The stiffening element can be made of any appropriate material that is preferably able to maintain the curved portion of the needle relatively straight while within the endoscope, such as spring steel. In some embodiments, the curved tool itself could have a stiffening sheath on the OD of the shaft that keeps the curved portion straightened until it is advanced beyond the sheath. The curved tool could also have an element in the lumen of the shaft that keeps the curved section straight until it is ready to be curved. When it is removed from the lumen the curved section of the tool returns to its curved unbiased shape. In some embodiments, two or more of the above bracing elements may be used.

### Curved Needle Driver Tool

One example of a curved endoscopic tool is a curved needle driver. In one embodiment, the needle driver includes an elongate shaft with a curved distal section. The end effector is preferably a hollow needle. A lumen preferably runs down the length of the tool, and a push rod that is in the lumen. There is a proximal handle that has one or more elements that can control both the advancement of the needle and the advancement of the pushrod separately or together. As shown in FIG. 20, the needle 1506 has a proximal portion 1510 and a curved distal tip portion 1508 in some embodiments. The needle 1506 is preferably hollow in some embodiments, and is configured to house a stylet 1512 that can contain, an element to de deployed, such as a fastener, preferably a T-tag fastener, flower tag fastener as described above, or other tissue anchor in some embodiments. The hollow curved needle 1506 in turn can be housed within a sheath 1504 as shown.

FIGS. 21-22 illustrate an endoscopic delivery system 1520 for actuating the curved needle driver 1502, according to one embodiment of the invention. FIG. 21 is a perspective view while FIG. 22 is a cut-away view. Sheath-holding element 1522, needle-advancing portion of the system 1524, and stylet-advancing element 1526 are illustrated. A portion of sheath, needle, and stylet are preferably held within securing elements within elements 1522, 1524, and 1526, respectively. Movement of needle-advancing portion 1524 relative to sheath-holding element 1522 in a distal direction will advance the curved needle 1506 out of the sheath 1504. Movement of stylet-advancing element 1526 relative to needle-advancing portion of the system 1524 in a distal direction will advance the stylet 1512 housing a fastening element (not shown) (e.g., when advancing a T-tag or flower petal fastener for attachment to the serosal surface of a wall of the GI tract). The sheath 1506 and curved needle 1506 including distal portion 1508 is also shown. The double wavy lines at reference point 1530 indicates that the sheath 1504 is shown abbreviated and is not to scale relative to components 1522, 1524, and 1526. In some embodiments, the sheath 1504, needle 1506, and/or stylet 1512 can be at least about 10 cm, 20 cm, 30 cm, 40 cm, 50 cm, 60 cm, 70 cm, 80 cm, 90 cm, 100 cm, or more in length. The cut-away view of the endoscopic delivery system in FIG. 22 further illustrates retainer tubing 1536 surrounding sheath 1504 to couple the sheath-holding element 1522 to the sheath 1504. Retainer tubing 1538 is also illustrated surrounding needle 1506 to couple the needle-advancing portion 1524 to the needle 1506. Furthermore, retainer tubing 1540 is illustrated surrounding stylet 1512 to couple the stylet-advancing element 1526 to the stylet 1512. In some embodiments, a needle driver has a stylet having a length greater than that of the needle, which in turn has a length greater than that of the sheath, consistent with the delivery system illustrated in FIGS. 21-22. In some embodiments, the stylet has a length of between about 45-85 inches, such as between about 52-72 inches, or between about 57-67 inches; the needle has a length of between about 40-80 inches, such as between about 47-67 inches, or between about 52-62 inches; and the sheath has a length of between about 38-78 inches, such as between about 45-65 inches, or between about 50-60 inches. In one embodiment, the stylet has a length of about 62 inches, the needle is about 57 inches, with a curved distal tip length of about 1.5 inches, and the sheath is about 55 inches in length. In some embodiments, the distal portion of the needle has a length of between about 0.5-2.5 inches, such as between about 1-2 inches, and when in its fully unstressed state, forms an arc of between about 30 to 80 degrees, such as between about 45 to 65 degrees, or about 55 degrees in some embodiments. The curved distal tip length can be no more than about 10%, 7%, 5%, 3%, or less of the total length of the needle in certain embodiments. The needle can have a point bevel arc of between about 10-30 degrees, 15-25 degrees, or about 23 degrees in some embodiments.

The needle with curved distal portion can advantageously be configured to penetrate a tissue wall with a desired trajectory. The arc of the distal portion of the needle can be adjusted by the operator as desired by actuating the needle driver an appropriate distance either out or back into the working channel of the endoscope, providing the operator with a degree of freedom in moving the needle to a desired location. For example, if the distal portion of the needle has an arc of 55 degrees in its fully unstressed state, pulling half of the length of the distal portion back into the working channel can result in a lesser arc of about 27.5 degrees. Rotation of the needle driver in an appropriate direction provides an additional degree of freedom.

### Multi-Stage Push Rod Deployment for Deploying Double-Sided Fasteners

In some embodiments, as illustrated in FIG. 21, the control on the handle 1528 that advances and retracts the push rod has multiple stops 1532, 1534, such as at least about two, three, four, or more stops to limit advancement in stages in one embodiment. For example, a two stop design 1532, 1534 as shown would allow the push rod to be advanced in two stages. One area where this would be beneficial is in delivering a double sided fastener, such as a t-tag, or multiple fasteners.

Aside from the benefits in viewing and targeting an area as shown above, another potential benefit of a curved tool for needle driving is that there is a more optimal angle of attack to pierce or penetrate the tissue wall. With a needle driver that is co-axial with the working channel of the endoscope, advancement of the needle takes an acute angle of attack to a wall of tissue if the endoscope is in the same lumen. With the curved needle, the angle of attack is closer to a right angle, and so the force required to pierce or penetrate the tissue could be less than with an acute angle. In some embodiments, the angle of curvature of the distal end portion is between about 45-135 degrees, preferably between about 60-120 degrees, or between about 75-115 degrees in some embodiments.

### Method of Use

Methods of using the various endoscopic delivery components described above, according to some embodiments, will now be disclosed. While the delivery components may be described below as being used together to attach a bypass sleeve with an attachment cuff to a wall of the gastrointestinal tract, it will be understood that the components could be used together for a variety of other applications; each component could be used separately for a variety of indications as well.

In some embodiments, a device used for creation of a working space in a body lumen, such as the stent described can be used to hold another object against the wall of the lumen, such as a cuff or one or more devices to be attached to the wall of the lumen. In some embodiments, the lumen is in the proximal esophagus, mid-esophagus, distal esophagus, gastroesophageal junction, stomach, such as the cardia of the stomach, pylorus, duodenum, jejunum, ileum, colon, or biliary tree.

Placing the end of the stent with the greater diameter facing proximally (toward an endoscope and the oral cavity), an object to be attached against the wall of the lumen of the gastroesophageal junction can be presented against the wall of the lumen and oriented where it is easier to target with an endoscope. The control catheter is running up the esophagus and out the patient's mouth. The stent controls are manipulated by the endoscopist.

The space creator in one embodiment can be used to facilitate endoscopic placement of tissue anchors through a cuff as described as described herein. In some embodiments, the space creator is used with the curved needle driver and expandable tag fastener disclosed herein as follows to attach a gastrointestinal bypass sleeve with an attachment cuff transmurally through the wall of the GEJ. In some embodiments, other fasteners, e.g., a T-pledget, button-shaped element, or any other fastener or other device, such as those disclosed in the Dann '605 application and other applications herein incorporated by reference, can be used when configured to be constrained in a hollow needle in a low crossing profile configuration, that can later be deployed out of the needle in an expanded configuration.

A fabric cuff including a first plurality of apertures with reinforcing rings configured to receive anchors for attaching a device is attached to the outside of the space creating stent with a suture that interlaces the struts of the stent with the cuff, such as through a proximal set of eyelets as described previously in the application.

A control catheter as described above is attached to the proximal end of the stent. Because the distal part of the stent is constrained in the fabric cuff, the proximal portion of the stent forms a funnel shape, with the proximal diameter of the stent greater than the distal diameter of the stent when in its relaxed state and the control catheter has a control element which controls the opening and closing of the proximal, larger, end of the funnel through a suture that goes through the loops at proximal part of the stent as described above. Actuating the control catheter in an appropriate direction, such as pulling the control handle, collapses the stent and advancing the control relaxes the tension in the suture and allows the stent to expand.

As illustrated in FIG. 23, the control catheter 1106, stent 1100 in a collapsed configuration, and cuff 1300 are advanced through an esophageal overtube (not shown for clarity) to a desired location, such as the gastroesophageal junction 1500 as shown. Also shown is a gastrointestinal bypass sleeve 100 attached to the distal end of the cuff 1300. In some embodiments, the gastrointestinal bypass sleeve is first inverted into a delivery catheter (not shown), delivered to the pylorus, and then delivered toposcopically into the intestine. Additional details regarding toposcopic delivery of a gastrointestinal sleeve 100 may be as described, for example, in U.S. patent application Ser. No. 11/861,156 filed Sep. 25, 2007, and hereby incorporated by reference in its entirety. More specifically, for example, FIGS. 1A-2E of the 11/861,156 application and the accompanying text at paragraphs [0054] to [0064] disclose various embodiments of toposcopic sleeves; FIG. 15H and the accompanying text at paragraph [0143] disclose an embodiment of a filling catheter and sleeve kit; and FIGS. 3A-16B and the accompanying text at paragraphs [0065] to [0142] and [0144] to [0150] disclose various toposcopic delivery systems and components including collapsible and steerable filling catheters, guidewires, techniques for occluding the distal end of the sleeve, and loop snares, all of which can be used or modified for use with the systems and methods described herein. After eversion of the sleeve, the sleeve can then be retracted proximally to, for example, to the gastroesophageal junction for attachment transmurally to the wall of the GEJ as described herein.

Once in place, the control 1222 (shown in FIG. 4) of the control catheter 1106 is advanced to release the tension in the control suture 1102 (not shown) which allows the proximal end 1014 of the stent 1100 to expand, as illustrated in FIG. 24. This expansion opens up the proximal end 1301 of the cuff 1300 similar to the opening of a flower and presents the intended anchor sites, which can be reinforced apertures 1302 in the cuff 1300 as described, for example in the '074 publication, so that they are more accessible to an endoscope coming down the lumen.

In one embodiment, the reinforced apertures are struts are made of polyurethane (pelethane) material and are attached to the cuff at multiple suture points. These struts act like ribs to give the cuff resistance to inversion without interfering with radial compliance. The struts can also be sutured in with vertical sutures to give additional radial compliance.

An endoscope 1500 in the lumen is positioned proximal to the cuff 1300, as shown in FIG. 25, and a curved needle driver 1502 or other anchor deploying or endoscopic suturing means can be used to suture the cuff 1300 to the wall of the lumen. If using the curved needle driver 1502 as described above, this is then advanced down a working channel of the endoscope 1500.

Under direct visualization the needle driver 1502 is advanced until the sheath is visible in the field of the endoscope 1500, as shown in FIG. 26. The endoscope 1500 and needle driver are then manipulated to cannulate the anchor hole in the cuff, as described in U.S. Provisional Application No. 60/943,304, previously incorporated by reference in its entirety. The needle 1502 is then advanced through the sheath through the wall of the lumen. The plunger on the needle driver is advanced to push out the anchor on the serosal side of the tissue, as described in U.S. Provisional Application Nos. 60/943,304 and 61/033,385, previously incorporated by reference in their entireties.

Next, the needle driver 1502 first is inserted through an aperture 1302 of an attachment cuff 1300, and out the other side of the aperture 1302. The curved needle 1502 can then cannulate the mucosal surface of the tissue wall at the GEJ, then exit the wall on the serosal surface.

The pushrod control of the needle driver, such as described above in connection with FIGS. 21-22 is then advanced to a first stop position within the working channel to eject the distal retention element 2000, as shown in FIG. 27. The needle 1502 is then withdrawn to the location of the second (more proximal) retention element 2104, as shown in FIG. 28. The pushrod control is then advanced to a second stop position within the endoscopic working channel to eject the proximal retention element 2106, as illustrated in FIG. 29. In other embodiments, however, if the proximal retention element 2104 is left outside of the needle 1502 or other delivery cannula (such as illustrated in FIG. 19) only a single stop pushrod control would be required.

The needle driver is then retracted and the anchoring process is repeated to place retention elements for each retention target on the cuff. Once the cuff is sutured in place, the suture loop attaching the stent to the cuff is mechanically cut, electrolytically detached, cauterized, etc., and the stent is collapsed and removed, leaving the cuff anchored to the luminal wall.

A perspective schematic view of one embodiment of the fastener system in use is shown in FIG. 30, with the distal retention element 2000 with plurality of petals 2006 connected to hub 2008 bearing against the serosal surface 1382 of a wall 1381 of the gastrointestinal junction. Proximal retention element 2104 is shown operably connected to an interior surface of the attachment cuff 1300 resting near mucosal surface 1380 of the wall 1381. Tension element 2012 is also illustrated as a dotted line.

The steps involving the curved needle driver 1502 could be repeated as many times as necessary if it is desired to anchor a device with more than one fastener system. Also, while the procedure may be performed under laparoscopic assistance, to further visualize and adjust the distal retention element from the serosal side of the tissue to be cannulated, one of ordinary skill in the art will appreciate that an endoscopic approach alone may be sufficient.

While delivery has been described in terms of transmurally delivering a distal retention element perorally from within the lumen of the esophagus to the serosal surface of the tissue wall at the gastroesophageal junction, and the proximal retention element on the mucosal side of the tissue wall inside of an attachment cuff, one of ordinary skill in the art will recognize that the fastener system can be used to fasten a wide range of devices to any appropriate tissue or organ. While described in terms of retaining a device through a transmural tissue wall, the fastening system may be also used, for example, to deploy retention elements on either side of one or more plications as well.

### Additional Methods

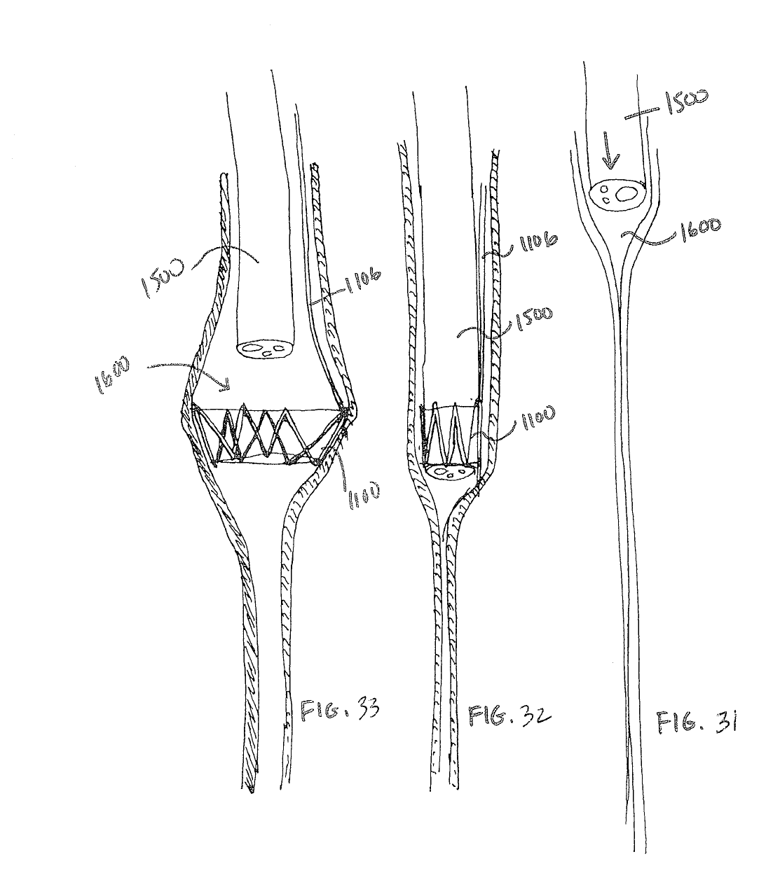

In another embodiment, illustrated in FIGS. 31-33, another method of placing a space-creating device is shown within a body lumen. FIG. 31 illustrates an endoscope 1500 being advanced distally (in the direction of arrow) into a body lumen 1600. Next, as shown in FIG. 32, a stent 1100 with attached control catheter 1106 such as described above is advanced over the endoscope 1500. The stent 1100 is then expanded proximally (and optionally distally as shown) in FIG. 33, and the endoscope 1500 is retracted partially to form the working channel 1600. Stents 1100 as illustrated in the method steps above are schematically illustrated for simplicity; stents 1100 as illustrated in e.g., FIG. 3C, other embodiments described herein, as well as conventional Z-stents are also contemplated with the methods disclosed herein.

In other embodiments, the space creator could be used in natural orifice surgeries. These procedures involve accessing the body cavity through a natural orifice such as the mouth, anus or vagina. In these procedures the natural body cavity wall is traversed by an instrument to gain access to the internal organs or other targets for specific therapies, such as for the ligation of fallopian tubes or oopherectomy. Disclosed are possible non-limiting ways of how a space creator could be used in these procedures.

### Transgastric Surgery

In this example, the space creator is a larger version of the stent described above for the gastroesophageal junction. It is approximately the size of a distended stomach, having a diameter of between about 3-12 cm, or 5-10 cm in some embodiments. The stent is collapsed and placed through an overtube into the stomach. It is then expanded creating an expanded working space in the stomach with the stomach wall under some tension. The tension is sufficient so that if the abdomen is insufflated with a laparoscopic trocar the stent has sufficient column strength to keep the stomach expanded.

An endoscope is then advanced into the stomach. The space creator makes a stable working space so a specific location to transect the wall of the stomach can be identified and accurately targeted. The space creator advantageously eliminates the need for air or CO2 insufflation to create and maintain a working space. This is potentially a simpler and more consistent method for space creation, as there is no need to prevent leakage of the insufflating gas. The dimensions of the space can remain relatively constant, without having to rely on a regulated pressure system to maintain the space.

The desired location can be determined through the use of, for example, fluoroscopy, transabdominal ultrasound, or endoscopic ultrasound. With regard to endoscopic ultrasound, this could facilitate a number of procedures. An endoscopic ultrasound device could be used in some embodiments to target the wall of the stomach so the point where the wall is traversed is most proximate to a target, for example, the gallbladder, liver, pancreas, kidneys, inferior vena cava, aorta, or other organ. One example in which this could prove beneficial is for targeting the liver or other organ for biopsy.

Once the incision is made in the wall of the stomach and the working instruments are through the wall, the space creating device can be collapsed to allow the stomach to return to its relaxed shape and give the instruments (e.g., laparoscopic instruments) more working space on the outside of the stomach. Two or more points of the control may need to be utilized with a larger stent design such as described above. The method of control could be the same or similar to that described above with multiple wires or sutures.

While this invention has been particularly shown and described with references to embodiments thereof, it will be understood by those skilled in the art that various changes in form and details may be made therein without departing from the scope of the invention. For all of the embodiments described above, the steps of the methods need not be performed sequentially. Further, the disclosure herein of any particular feature in connection with an embodiment can be used in all other disclosed embodiments set forth herein.

We claim:

1. An expandable fastener for securing a device transmurally to a surface of a tissue wall, the fastener comprising:
a first retention element comprising a plurality of petals extending from a central hub; wherein the plurality of petals has a total surface area; wherein the first retention element is movable from a compressed configuration for delivery to the surface of the tissue wall and an expanded configuration for engaging tissue; wherein the first retention element defines an effective footprint of the first retention element, wherein the effective footprint is defined by the smallest diameter circle circumscribing the plurality of petals while the first retention element is in its expanded configuration; wherein the total surface area of the plurality of petals is no more than about 80% of the area of the effective footprint of the first retention element.

2. The expandable fastener of claim 1, wherein the surface area of the plurality of petals is no more than about 70% of the area of the effective footprint of the first retention element.

3. The expandable fastener of claim 1, wherein the surface area of the plurality of petals is no more than about 60% of the area of the effective footprint of the first retention element.

4. The expandable fastener of claim 1, further comprising a tension element having an elongate body, a proximal end, and a distal end, the tension element operably attached to the central hub.

5. The expandable fastener of claim 1, wherein the smallest diameter circle circumscribing the plurality of petals has a diameter of between about 0.10 inches and 0.50 inches.

6. The expandable fastener of claim 1, wherein the first retention element comprises between 2 and 10 petals.

7. The expandable fastener of claim 1, wherein the plurality of petals is formed from one or more wires, the one or more wires having a diameter of between about 0.001 inch and 0.050 inches.

8. The expandable fastener of claim 1, wherein the plurality of petals comprise a tissue-ingrowth material.

9. The expandable fastener of claim 1, wherein the tension element has a length that is at least about 100% of the thickness of the tissue wall.

10. The expandable fastener of claim 1, further comprising a second retention element operably connected to the proximal end of the tension element.

11. The expandable fastener of claim 10, wherein the tension element comprises a suture.

12. The expandable fastener of claim 10, wherein the second retention element comprises a T-tag.

13. The expandable fastener of claim 10, wherein the second retention element comprises a button.

14. The expandable fastener of claim 1, wherein each of the plurality of petals is configured to be independently movable with respect to the other petals.
